(19) United States
(12) Patent Application Publication
(10) Pub. No.: US 2026/0218206 A1
(43) Pub. Date: Jul. 30, 2026

(54) GENE ENCODING CYTOCHROME P450 AND USE THEREOF

(71) Applicant: KUMIAI CHEMICAL INDUSTRY CO., LTD., Tokyo (JP)

(72) Inventors: Shunsuke YAMAMOTO, Tokyo (JP); Kiyoshi KAWAI, Tokyo (JP)

(73) Assignee: KUMIAI CHEMICAL INDUSTRY CO., LTD., Tokyo (JP)

(21) Appl. No.: 19/652,185

(22) Filed: Apr. 20, 2026

Related U.S. Application Data
(62) Division of application No. 18/009,795, filed on Jun. 24, 2021, now Pat. No. 12,630,829.

(30) Foreign Application Priority Data
Jun. 26, 2020 (JP) 2020-110269
Aug. 7, 2020 (JP) 2020-134782

(51) Int. Cl. C12N 15/52; C12N 9/02; C12N 15/82

(52) U.S. Cl. CPC C12N 15/52; C12N 9/0079; C12N 15/8243; C12Y 114/15004

(57) ABSTRACT

Metabolic activity is exhibited against a larger number of compounds having different acting properties. A cytochrome P450 gene encodes a protein having an amino acid sequence of any of SEQ ID NOS: 2, 4, 6, and 8.

### CROSS-REFERENCE TO RELATED APPLICATIONS

This application is a Divisional of copending application Ser. No. 18/009,795, filed on Dec. 12, 2022, which is the National Phase under 35 U.S.C. § 371 of International Application No. PCT/JP2021/024038, filed on Jun. 24, 2021, which claims the benefit under 35 U.S.C. § 119(a) to Patent Application No. 2020-110269, filed in Japan on Jun. 26, 2020, and Patent Application No. 2020-134782, filed in Japan on Aug. 7, 2020, all of which are hereby expressly incorporated by reference into the present application.

### REFERENCE TO SEQUENCE LISTING

The application contains a Sequence Listing which has been submitted electronically in .XML format and is hereby incorporated by reference in its entirety. Said .XML copy, created on Apr. 17, 2026, is named “2026-04-17_SequenceListing 1254-0666PUS2.xml” and is 64,607 bytes in size. The sequence listing contained in this .XML file is part of the specification and is hereby incorporated by reference herein in its entirety.

### TECHNICAL FIELD

The present invention relates to a gene encoding a cytochrome P450 having a characteristic substrate specificity, and use thereof.

### BACKGROUND ART

Cytochrome P450 (hereinafter P450 or CYP) is a group of hemoproteins that are present in a wide range of species including microorganisms, plants, and animals. P450 has monooxygenase activity as its function, and there are a large number of molecular species having various substrate specificities. There are 57 molecular species of P450 in humans, and as for plants, there are about 250 molecular species in Arabidopsis thaliana, and about 450 molecular species in a rice plant. P450 is known as an enzyme involved mainly in drug metabolism in humans, and involved in secondary metabolism or drug metabolism in plants.

A P450 involved in drug metabolism in humans catalyzes, as each of the molecular species, a large number of substrates having different acting properties, and on the other hand, it has been revealed that a plant P450 has very high substrate specificity, and recognizes and metabolically degrades only limited compounds having limited acting properties (Non Patent Literature 1: Hideo Ohkawa and Hideyuki Inui, Pest Manag Sci 2015; 71: 824-828). In particular, there are a very small number of P450s specified to be involved in drug metabolism in crops. That is, CYP71A10 of soybean, CYP71C6v1 of wheat (Non Patent Literature 2: Balazs Siminszky, Phytochem Rev (2006) 5:445-458), and CYP81A6 (Non Patent Literature 3: Gang Pan et al, Plant Mol Biol (2006) 61:933-943) and CYP72A31 (Non Patent Literature 4: Hiroaki Saika et al, Plant Physiology (2014) Vol. 166, pp. 1232-1240) of a rice plant, and there are a very limited number of drugs that can be metabolically degraded by these P450s. Specifically, it has been revealed that CYP71A10, CYP71C6v1, and CYP72A31 respectively metabolically degrade a photosystem II inhibitor such as chlorotoluron, an ALS inhibitor such as chlorsulfuron, and an ALS inhibitor such as bispyribac-sodium salt.

In particular, as for CYP81A6 of a rice plant disclosed in Non Patent Literature 3, it is disclosed that CYP81A6 has substrate specificity to bentazone and sulfonylurea, and that a transformed plant having resistance to bentazone and sulfonylurea is produced by using CYP81A6 (Patent Literature 1: U.S. Pat. No. 8,049,063 B2). Besides, Non Patent Literature 5 (The 26th Asian-Pacific Weed Science Society Conference) discloses that CYP81A6 is involved in metabolism of fenquinotrione.

### CITATION LIST

### Non Patent Literature

Non Patent Literature 1: Hideo Ohkawa and Hideyuki Inui, Pest Manag Sci 2015; 71: 824-828
Non Patent Literature 2: Balazs Simiznszky, Phytochem Rev (2006) 5:445-458
Non Patent Literature 3: Gang Pan et al, Plant Mol Biol (2006) 61:933-943
Non Patent Literature 4: Hiroaki Saika et al, Plant Physiology (2014) Vol. 166, pp. 1232-1240
Non Patent Literature 5: The 26th Asian-Pacific Weed Science Society Conference program book (2017) pp. 232

### PATENT LITERATURE

Patent Literature 1: U.S. Pat. No. 8,049,063 B2

### SUMMARY OF INVENTION

### Technical Problem

To date, no plant P450s has been known, however, to have metabolic activity against a large number of compounds. Therefore, in consideration of the above-described circumstances, an object of the present invention is to provide a gene encoding a novel cytochrome P450 having metabolic activity against a larger number of compounds as compared to known P450s, and use thereof.

### Solution to Problem

The present inventors made earnest studies to solve the above-described problem, and as a result, have succeeded in identifying a gene encoding a cytochrome P450 having metabolic activity against a variety of compounds having different acting properties against plants, and thus, the present invention was accomplished.

Specifically, the present invention encompasses the following:

(1) A cytochrome P450 gene encoding a protein described in any of the following (a) to (c):
(a) a protein comprising an amino acid sequence of any of SEQ ID NOS: 2, 4, 6, and 8;
(b) a protein comprising an amino acid sequence having 80% or more identity to the amino acid sequence of any of SEQ ID NOS: 2, 4, 6, and 8, and having metabolic activity against a microtubule assembly inhibitor classified as code 3 of Herbicide Resistance Action Committee (HRAC) classifications, a carotenoid biosynthesis inhibitor in phytoene desaturase (PDS) classified as code 12 of HRAC classifications, a protoporphyrinogen oxidase inhibitor classified as code 14 of HRAC classifications, and a dihydropteroate synthase inhibitor classified as code 18 of HRAC classifications; and
(c) a protein comprising an amino acid sequence encoded by a polynucleotide that hybridizes, under stringent conditions, with a polynucleotide consisting of a nucleotide sequence complementary to a nucleotide sequence of any of SEQ ID NO: 1, 3, 5, and 7, and having metabolic activity against a microtubule assembly inhibitor classified as code 3 of HRAC classifications, a carotenoid biosynthesis inhibitor in phytoene desaturase (PDS) classified as code 12 of HRAC classifications, a protoporphyrinogen oxidase inhibitor classified as code 14 of HRAC classifications, and a dihydropteroate synthase inhibitor classified as code 18 of HRAC classifications.

(2) The cytochrome P450 gene according to (1), wherein the protein consists of an amino acid sequence of (d) or (e):
(d) an amino acid sequence of any of SEQ ID NOS: 10, 12, 14, and 16; or
(e) an amino acid sequence having 80% or more identity to the amino acid sequence of any of SEQ ID NOS: 10, 12, 14, and 16.

(3) The cytochrome P450 gene according to (1), wherein the protein has metabolic activity against an acetolactate synthase inhibitor classified as code 2 of HRAC classifications, a microtubule assembly inhibitor classified as code 3 of HRAC classifications, a photosystem II inhibitor classified as code 6 of HRAC classification, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications.

(4) An expression vector comprising the cytochrome P450 gene according to any of (1) to (3).

(5) A transformant comprising the expression vector according to (4).

(6) A transgenic plant comprising the expression vector according to (4).

(7) The transgenic plant according to (6), wherein the plant is a plant body, a plant organ, a plant tissue, or a cultured plant cell.

(8) A method for producing a plant having resistance to a microtubule assembly inhibitor classified as code 3 of HRAC classifications, a carotenoid biosynthesis inhibitor in phytoene desaturase (PDS) classified as code 12 of HRAC classifications, a protoporphyrinogen oxidase inhibitor classified as code 14 of HRAC classifications, and a dihydropteroate synthase inhibitor classified as code 18 of HRAC classifications, the method comprising culturing or cultivating the transgenic plant according to (6) or (7).

(9) A method for controlling weed noxious to the transgenic plant according to (6) or (7), comprising treating a field used for cultivating the transgenic plant with at least one inhibitor selected from the group consisting of an acetolactate synthase inhibitor classified as code 2 of HRAC classifications, a microtubule assembly inhibitor classified as code 3 of HRAC classifications, a photosystem II inhibitor classified as code 6 of HRAC classification, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications.

(10) A transformation method comprising: a step of introducing, into a host cell, an expression vector comprising the cytochrome P450 gene according to any of (1) to (3), and an additional gene; and a step of selecting, as a transformant, a cell that grows in the presence of at least one inhibitor selected from the group consisting of an acetolactate synthase inhibitor classified as code 2 of HRAC classifications, a microtubule assembly inhibitor classified as code 3 of HRAC classifications, a photosystem II inhibitor classified as code 6 of HRAC classification, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications.

The present specification encompasses the disclosed contents of JP Patent Application No. 2020-110269 and JP Patent Application No. 2020-134782, based on which the present application claims the benefit of priority.

### Advantageous Effects of Invention

According to the present invention, a cytochrome P450 gene having metabolic activity against a large number of drugs having different acting properties can be provided. When the cytochrome P450 gene of the present invention is used, resistance to the large number of drugs having different acting properties can be imparted to plants. Besides, when the cytochrome P450 gene of the present invention is used as a selection marker gene, a totally novel transformation method can be constructed. In addition, the cytochrome P450 gene of the present invention can be used as a resistance marker gene serving as an index of resistance to a large number of drugs having different acting properties.

### BRIEF DESCRIPTION OF DRAWINGS

FIG. 1 is a diagram illustrating results of multiple alignment analysis of CYP81A6 (SEQ ID NO: 39), AK375492 (SEQ ID NO: 16), and AK369081 (SEQ ID NO: 14).

FIG. 2 is a diagram illustrating results of multiple alignment analysis of CYP81A6 (SEQ ID NO: 39), KJ541960 (SEQ ID NO: 12), and AK454412 (SEQ ID NO: 10).

FIG. 3 is an electrophoresis photograph of a result of PCR using a primer set for amplifying AK454412 gene.

FIG. 4 is a characteristic diagram illustrating production process of E. coli expression construct of AK454412 gene.

FIG. 5 is a characteristic diagram illustrating production process of E. coli expression construct of AJ303373 gene.

FIG. 6 is a SDS-PAGE photograph illustrating expression of AK454412 protein and TaCPR protein.

FIG. 7-1 is a characteristic diagram illustrating a result of LC/MS analysis performed on a negative control plot (empty vector expression crude enzyme reaction plot).

FIG. 7-2 is a characteristic diagram illustrating a result of LC/MS analysis performed on an AK454412-TaCPR co-expression crude enzyme reaction plot.

FIG. 8 is a characteristic diagram illustrating production process of an AK454412 gene forced expression binary vector.

FIG. 9 illustrates photographs of verification results on fenquinotrione resistance in wild type Arabidopsis thaliana and Arabidopsis thaliana having AK454412 forced expression.

FIG. 10-1 is a table showing verification results on resistance to various herbicides in wild type Arabidopsis thaliana and Arabidopsis thaliana having AK454412 forced expression.

FIG. 10-2 is a table showing verification results on resistance to various herbicides in wild type Arabidopsis thaliana and Arabidopsis thaliana having AK454412 forced expression.

FIG. 10-3 is a table showing verification results on resistance to various herbicides of wild type Arabidopsis thaliana and Arabidopsis thaliana having AK454412 forced expression.

FIG. 10-4 is a table showing verification results on resistance to various herbicides of wild type Arabidopsis thaliana and Arabidopsis thaliana having AK454412 forced expression.

FIG. 11 is a characteristic diagram illustrating results of expression analysis of AK454412 gene in various wheats.

FIG. 12 is an electrophoresis photograph illustrating a result of PCR using a primer set for amplifying KJ541960 gene.

FIG. 13 is a characteristic diagram illustrating production process of a KJ541960 gene expression vector.

FIG. 14 is a characteristic diagram illustrating a result of in vitro drug metabolism test of KJ541960 protein performed against fenquinotrione.

FIG. 15 is a characteristic diagram illustrating production process of AK375492 and AK369081 gene expression vectors.

FIG. 16 is a SDS-PAGE photograph illustrating expression of AK375492 and AK369081 proteins and TaCPR protein.

FIG. 17 is a characteristic diagram illustrating a result of in vitro drug metabolism test of AK369081 protein performed against fenquinotrione.

### DESCRIPTION OF EMBODIMENTS

The present invention will now be described in detail.

### 1. Cytochrome P450 Gene

A cytochrome P450 gene according to the present invention (hereinafter sometimes simply referred to as the P450 gene) is a gene having a prescribed amino acid sequence, and encoding a P450 having metabolic activity against a large number of drugs having different acting properties. The P450 gene of the present invention has certain sequence similarity, at the amino acid level, to CYP81A6 of a rice plant, but is different therefrom in encoding a P450 having metabolic activity against a wider range of compounds than CYP81A6.

Generally, a protein classified as P450 has a structure including a transmembrane region on the N-terminal side, and an active region continuously to the transmembrane region. The P450 gene of the present invention may encode a protein comprising an amino acid sequence of a region involved in the metabolic activity excluding a transmembrane sequence, or may encode a protein comprising an amino acid sequence including the transmembrane region and the region involved in the metabolic activity.

In the P450 gene of the present invention, examples of an amino acid sequence of the region involved in the metabolic activity (namely, an amino acid sequence excluding the transmembrane sequence) include amino acid sequences of SEQ ID NOS: 2, 4, 6, and 8.

Here, the amino acid sequence of SEQ ID NO: 2 is an amino acid sequence of a wheat-derived P450 registered in GenBank as accession No. AK454412 excluding a transmembrane sequence, and is encoded by a nucleotide sequence of SEQ ID NO: 1. Besides, the amino acid sequence of SEQ ID NO: 4 is an amino acid sequence of a wheat-derived P450 registered in GenBank as accession No. KJ541960 excluding a transmembrane sequence, and is encoded by a nucleotide sequence of SEQ ID NO: 3. The amino acid sequence of SEQ ID NO: 6 is an amino acid sequence of a barley-derived P450 registered in GenBank as accession No. AK369081 excluding a transmembrane sequence, and is encoded by a nucleotide sequence of SEQ ID NO: 5. The amino acid sequence of SEQ ID NO: 8 is an amino acid sequence of a barley-derived P450 registered in GenBank as accession No. AK375492 excluding a transmembrane sequence, and is encoded by a nucleotide sequence of SEQ ID NO: 7. These P450s consisting of the amino acid sequences set forth in SEQ ID NOS: 2, 4, 6, and 8 (excluding the transmembrane region) are regions involved in the metabolic activity against a large number of drugs having different acting properties.

The amino acid sequence of SEQ ID NO: 4 (KJ541960) has 96% sequence identity, the amino acid sequence of SEQ ID NO: 6 (AK369081) has 80% sequence identity, and the amino acid sequence of SEQ ID NO: 8 (AK375492) has 93% sequence identity to the amino acid sequence of SEQ ID NO: 2 (AK454412). Besides, the nucleotide sequence of SEQ ID NO: 3 (KJ541960) has 96% sequence identity, the nucleotide sequence of SEQ ID NO: 5 (AK369081) has 85% sequence identity, and the nucleotide sequence of SEQ ID NO: 7 (AK375492) has 94% sequence identity to the nucleotide sequence of SEQ ID NO: 1 (AK454412).

The P450 gene of the present invention is not limited to those specified by SEQ ID NOS: 1 to 8, and, for example, may be a gene encoding a protein having an amino acid sequence having 80% or more, preferably 85% or more, more preferably 90% or more, and most preferably 95% or more sequence identity to the amino acid sequence of SEQ ID NO: 2, 4, 6, or 8, and having metabolic activity against a large number of drugs having different acting properties. A value of sequence identity can be calculated by BLASTN or BLASTX program implementing BLAST algorithm (default settings). It is noted that a value of sequence identity is calculated as a ratio, in the total number of all compared amino acid residues, of the number of completely matching amino acid residues calculated in pairwise alignment analysis of a pair of amino acid sequences.

Besides, the P450 gene of the present invention is not limited to those specified by SEQ ID NOS: 1 to 8, and, for example, may be a gene encoding a protein having an amino acid sequence obtained by substituting, deleting, inserting, or adding one or several amino acids in the amino acid sequence of SEQ ID NO: 2, 4, 6, or 8, and having metabolic activity against a large number of drugs having different acting properties. Here, several amino acids refer to, for example, 2 to 50, preferably 2 to 40, more preferably 2 to 30, more preferably 2 to 20, further preferably 2 to 10, and most preferably 2 to 5 amino acids.

Furthermore, the P450 gene of the present invention is not limited to those specified by SEQ ID NOS: 1 to 8, and, for example, may be a gene encoding a protein that hybridizes, under stringent conditions, with all or some of complementary strands of a DNA consisting of the nucleotide sequence of any of SEQ ID NO: 1, 3, 5, and 7, and having metabolic activity against a large number of drugs having different acting properties. The term “stringent conditions” used herein means conditions where what is called a specific hybrid is formed but a non-specific hybrid is not formed, and the conditions can be appropriately determined referring to, for example, Molecular Cloning: A Laboratory Manual (Third Edition). Specifically, stringency can be set in accordance with a temperature and a salt concentration in a solution in Southern hybridization, and a temperature and a salt concentration in a solution in a washing step in Southern hybridization. In more detail, stringent conditions are, for example, a sodium concentration of 25 to 500 mM, and preferably 25 to 300 mM, and a temperature of 42 to 68° C., and preferably 42 to 65° C. More specifically, the conditions are 5×SSC (83 mM NaCl and 83 mM sodium citrate), and a temperature of 42° C.

As described above, it can be confirmed whether or not a gene consisting of a nucleotide sequence different from SEQ ID NOS: 1, 3, 5, and 7, or a gene encoding an amino acid sequence different from SEQ ID NOS: 2, 4, 6, and 8 functions as a cytochrome P450 to encode a protein having metabolic activity against a prescribed drug by producing a transformed plant using an expression vector obtained by integrating the gene between a Nos promoter derived from Agrobacterium tumefaciens and a terminator or the like, and examining whether or not the transformed plant can grow in the presence of the drug. It is noted that the drug can be appropriately selected from a large number of drugs having different acting properties described in detail below.

On the other hand, the P450 gene of the present invention may be a gene encoding a protein comprising an amino acid sequence comprising a transmembrane region and a region involved in metabolic activity for a drug. Examples of the amino acid sequence of the transmembrane region and the region involved in metabolic activity comprise amino acid sequences of SEQ ID NO: 10, 12, 14, and 16.

Here, the amino acid sequence of SEQ ID NO: 10 is an amino acid sequence comprising a transmembrane sequence in a wheat-derived P450 registered in GenBank as accession No. AK454412, and is encoded by a nucleotide sequence of SEQ ID NO: 9. Besides, the amino acid sequence of SEQ ID NO: 12 is an amino acid sequence comprising a transmembrane sequence in a wheat-derived P450 registered in GenBank as accession No. KJ541960, and is encoded by a nucleotide sequence of SEQ ID NO: 11. The amino acid sequence of SEQ ID NO: 14 is an amino acid sequence including a transmembrane sequence in a barley-derived P450 registered in GenBank as accession No. AK369081, and is encoded by a nucleotide sequence of SEQ ID NO: 13. The amino acid sequence of SEQ ID NO: 16 is an amino acid sequence including a transmembrane sequence in a barley-derived P450 registered in GenBank as accession No. AK375492, and is encoded by a nucleotide sequence of SEQ ID NO: 15.

It is noted that the amino acid sequence of SEQ ID NO: 2 described above is a partial sequence of the amino acid sequence of SEQ ID NO: 10. Similarly, the amino acid sequence of SEQ ID NO: 4 described above is a partial sequence of the amino acid sequence of SEQ ID NO: 12. Similarly, the amino acid sequence of SEQ ID NO: 6 described above is a partial sequence of the amino acid sequence of SEQ ID NO: 14. Similarly, the amino acid sequence of SEQ ID NO: 8 described above is a partial sequence of the amino acid sequence of SEQ ID NO: 16.

The amino acid sequence of SEQ ID NO: 12 (KJ541960) has 94% sequence identity, the amino acid sequence of SEQ ID NO: 14 (AK369081) has 79% sequence identity, and the amino acid sequence of SEQ ID NO: 16 (AK375492) has 90% sequence identity to the amino acid sequence of SEQ ID NO: 10 (AK454412). Besides, the nucleotide sequence of SEQ ID NO: 11 (KJ541960) has 95% sequence identity, the nucleotide sequence of SEQ ID NO: 13 (AK369081) has 85% sequence identity, and the nucleotide sequence of SEQ ID NO: 15 (AK375492) has 93% sequence identity to the nucleotide sequence of SEQ ID NO: 9 (AK454412).

The P450 gene of the present invention is not limited to those specified by SEQ ID NOS: 9 to 16, and, for example, may be a gene encoding a protein comprising an amino acid sequence having 80% or more, preferably 85% or more, more preferably 90% or more, and most preferably 95% or more sequence identity to the amino acid sequence of SEQ ID NO: 10, 12, 14, or 16, and having metabolic activity against a large number of drugs having different acting properties. A value of sequence identity can be calculated by BLASTN or BLASTX program implementing BLAST algorithm (default settings). It is noted that a value of sequence identity is calculated as a ratio, in the total number of all compared amino acid residues, of the number of completely matching amino acid residues calculated in pairwise alignment analysis of a pair of amino acid sequences.

Besides, the P450 gene of the present invention is not limited to those specified by SEQ ID NOS: 9 to 16, and, for example, may be a gene encoding a protein having an amino acid sequence obtained by substituting, deleting, inserting, or adding one or several amino acids in the amino acid sequence of SEQ ID NO: 10, 12, 14, or 16, and having metabolic activity against a large number of drugs having different acting properties. Here, several amino acids refer to, for example, 2 to 55, preferably 2 to 45, more preferably 2 to 35, more preferably 2 to 25, further preferably 2 to 15, and most preferably 2 to 5 amino acids.

Furthermore, the P450 gene of the present invention is not limited to those specified by SEQ ID NOS: 9 to 16, and, for example, may be a gene encoding a protein that hybridizes, under stringent conditions, with all or some of complementary strands of a DNA consisting of the nucleotide sequence of any of SEQ ID NO: 9, 11, 13, and 15, and having metabolic activity against a large number of drugs having different acting properties. The term “stringent conditions” used herein means conditions where what is called a specific hybrid is formed but a non-specific hybrid is not formed, and the conditions can be appropriately determined referring to, for example, Molecular Cloning: A Laboratory Manual (Third Edition). Specifically, stringency can be set in accordance with a temperature and a salt concentration in a solution in Southern hybridization, and a temperature and a salt concentration in a solution in a washing step in Southern hybridization. In more detail, stringent conditions are, for example, a sodium concentration of 25 to 500 mM, and preferably 25 to 300 mM, and a temperature of 42 to 68° C., and preferably 42 to 65° C. More specifically, the conditions are 5×SSC (83 mM NaCl and 83 mM sodium citrate), and a temperature of 42° C.

As described above, it can be confirmed whether or not a gene consisting of a nucleotide sequence different from SEQ ID NOS: 9, 11, 13, or 15, or a gene encoding an amino acid sequence different from SEQ ID NOS: 10, 12, 14, or 16 functions as a cytochrome P450 to encode a protein having metabolic activity against a prescribed drug by producing a transformed plant using an expression vector obtained by integrating the gene between a Nos promoter derived from Agrobacterium tumefaciens and a terminator or the like, and examining whether or not the transformed plant can grow in the presence of the drug. It is noted that the drug can be appropriately selected from a large number of drugs having different acting properties described in detail below.

The P450 of the present invention has metabolic activity against a large number of drugs having different acting properties as compared with conventional P450s, such as CYP81A6. Examples of the drugs against which the P450 of the present invention has metabolic activity include an acetolactate synthase inhibitor classified as code 2 of HRAC classifications, a microtubule assembly inhibitor classified as code 3 of HRAC classifications, a photosystem II inhibitor classified as code 6 of HRAC classifications, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications.

The P450 of the present invention is different from the conventionally known other P450s in having metabolic activity against a protoporphyrinogen oxidase inhibitor classified as code 14 of HRAC classifications, a carotenoid biosynthesis inhibitor in phytoene desaturase (PDS) classified as code 12 of HRAC classifications, a 4-hydroxyphenylpyruvate dioxygenase inhibitor classified as code 27 of HRAC classifications, a dihydropteroate synthase inhibitor classified as code 18 of HRAC classifications, and a microtubule assembly inhibitor classified as code 3 of HRAC classifications among those described above.

Here, an acetolactate synthase inhibitor means a compound exhibiting herbicidal activity by inhibiting acetolactate synthase. Examples of the acetolactate synthase inhibitor include sulfonylurea compounds such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfron, flupyrsulfron, foramsulfuron, halosulfuron-methyl, imazosulfuron, iodosulfuron, mesosulfuron, metsulfuron-methyl, nicosulfuron, oxasulfuron, primisulfuron, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, and tritosulfuron; imidazolinone compounds such as imazapic, imazamethabenz-methyl, imazamox, imazapyr, imazaquin, and imazethapyr; triazolopyrimidine compounds such as cloransulam-methyl, diclosulam, florasulam, flumetsulam, metosulam, and penoxsulam; pyrimidinyl (thio)benzoate compounds such as bispyribac-sodium salt, pyribenzoxim, pyriftalid, pyrithiobac-sodium salt, and pyriminobac-methyl; sulfonylaminocarbonyltriazolinone compounds such as flucarbazone-sodium salt, and propoxycarbazone-sodium salt; and sulphonanilide compounds such as pyrimisulfan and triafamone.

A photosystem II inhibitor classified as code 6 of HRAC classifications means a compound exhibiting herbicidal activity by inhibiting photosystem II involved in photosynthesis. Examples of the photosystem II inhibitor classified as code 6 of HRAC classifications include nitrile compounds such as bromophenoxim, bromoxynil, and ioxynil; benzothiadiazinone compounds such as bentazone; and phenylpyridazine compounds such as pyridate and pyridafol.

A protoporphyrinogen oxidase inhibitor means a compound exhibiting herbicidal activity by inhibiting protoporphyrinogen oxidase. Examples of the protoporphyrinogen oxidase inhibitor include diphenyl ether compounds such as acifluorfen, bifenox, chlomethoxynil (chlomethoxyfen), fluoroglycofen-ethyl, fomesafen, halosafen, lactofen, and oxyfluorfen; phenyl pyrazole compounds such as fluazolate and pyraflufen-ethyl; N-phenylphthalimide compounds such as cinidon-ethyl, flumioxazin, and flumiclorac-pentyl; thiadiazole compounds such as fluthiacet-methyl and thidiazimin; oxadiazole compounds such as oxadiazon and oxadiargyl; triazolinone compounds such as azafenidin, carfentrazone-ethyl, and sulfentrazone; oxazolidinedione compounds such as pentoxazone; pyrimidinedione compounds such as benzphendizone and butafenacil; phenylpyrazole compounds such as pyraflufen-ethyl and fluazolate; and pyraclonil, profluazole, and flufenpyr-ethyl.

A carotenoid biosynthesis inhibitor in phytoene desaturase (PDS) means a compound exhibiting herbicidal activity by inhibiting phytoene desaturase. Examples of the carotenoid biosynthesis inhibitor include pyridazinone compounds such as norflurazon; pyridinecarboxamide compounds such as diflufenican and picolinafen; and beflubutamid, fluridone, flurochloridone, and flurtamone.

A 4-hydroxyphenylpyruvate dioxygenase inhibitor means a compound exhibiting herbicidal activity by inhibiting 4-hydroxyphenylpyruvate dioxygenase. Examples of the 4-hydroxyphenylpyruvate dioxygenase inhibitor include triketone compounds such as fenquinotrione, mesotrione, and sulcotrione; isoxazole compounds such as isoxachlortole and isoxaflutole; pyrazole compounds such as benzofenap, pyrazolate (pyrazolynate), and pyrazoxyfen; and benzobicyclon.

A dihydropteroate synthase inhibitor means a compound exhibiting herbicidal activity by inhibiting dihydropteroate synthase. Examples of the dihydropteroate synthase include carbamate compounds such as asulam.

A microtubule assembly inhibitor is a compound exhibiting herbicidal activity by acting on a globular protein, tubulin to inhibit polymerization of microtubule. Examples of the microtubule assembly inhibitor include dinitroaniline compounds such as bethrodine (benfluralin), butralin, dinitramine, ethalfluralin, oryzalin, pendimethalin, and trifluralin; phosphoramidate compounds such as amiprophos-methyl and butamifos; pyridine compounds such as dithiopyr and thiazopyr; benzamide compounds such as propyzamide and tebutam; and benzoic acid compounds such as TCTP (chlorthal-dimethyl).

The P450 of the present invention has excellent metabolic activity particularly against pyrimisulfan, penoxsulam, bensulfuron-methyl, metsulfuron-methyl, imazaquin, nicosulfuron, sulfometuron-methyl, bentazone, oxadiargyl, sulfentrazone, pyraflufen-ethyl, fomesafen, diflufenican, picolinafen, fluridone, norflurazon, pyrazolate, benzofenap, fenquinotrione, asulam, and pendimethalin among the exemplarily described specific compounds.

### 2. Expression Vector

An expression vector of the present invention can be obtained by linking (inserting) the cytochrome P450 gene of the present invention to (into) an appropriate vector. The vector for inserting the cytochrome P450 gene of the present invention thereinto is not especially limited as long as it is replicable in a host, and examples include a plasmid, a shuttle vector, and a helper plasmid.

Examples of a plasmid DNA include plasmids derived from E. coli (such as pBR322, pBR325, pUC118, pUC119, pUC18, pCU19, and pBluescript), plasmids derived from Bacillus subtilis (such as pUB110 and pTP5), and plasmids derived from yeast (such as YEp13 and YCp50), and examples of a phage DNA include λ phages (Charon 4A, Charon 21A, EMBL3, EMBL4, λgt10, λgt11, and λZAP). Alternatively, an animal virus such as a retrovirus or vaccinia virus, or an insect virus vector such as baculovirus can be used.

For inserting the cytochrome P450 gene of the present invention into a vector, a method in which a DNA fragment comprising the cytochrome P450 gene is first purified, the purified DNA fragment is cleaved with an appropriate restriction enzyme, and the resultant is inserted into a restriction enzyme site or a multicloning site of an appropriate vector DNA to be linked to the vector, or the like is employed.

In the present invention, to express an arbitrary gene, the arbitrary gene can be further inserted into the expression vector. A method for inserting the arbitrary gene is the same as the method for inserting the cytochrome P450 gene of the present invention into a vector.

The cytochrome P450 gene of the present invention can be examined for the herbicide resistance after being linked between a Nos promoter derived from Agrobacterium tumefaciens and a terminator or the like to be introduced into a plant. Examples of the promoter include, in addition to a Nos promoter, cauliflower mosaic virus 35S promoter (CaMV35S), various actin gene promoters, various ubiquitin gene promoters, a nopaline synthase gene promoter, a tobacco PR1a gene promoter, a tomato ribulose 1,5-diphosphate carboxylase/oxidase small subunit gene promoter, a napin gene promoter, and an oleosin gene promoter. Among these, cauliflower mosaic virus 35S promoter, an actin gene promoter, or a ubiquitin gene promoter can be more preferably used.

In this manner, various vectors can be used in the present invention. Besides, a target arbitrary gene can be connected to the cytochrome P450 gene of the present invention in the sense or antisense direction, so that the resultant can be inserted into a vector designated as a binary vector, such as pBI101 (Clontech).

### 3. Production of Transformant

A transformant of the present invention can be obtained, for example, by introducing the above-described expression vector of the present invention into a host. Here, the host is not especially limited as long as it can express the cytochrome P450 gene of the present invention, and is preferably a plant. When the expression vector of the present invention is introduced into a host, the resistance to a large number of drugs having different acting properties described above can be obtained through the expression of the cytochrome P450 gene of the present invention. Therefore, it can be confirmed whether or not the above-described expression vector of the present invention has been able to be introduced into the host by evaluation using, as an index, the resistance to the drug. In other words, the cytochrome P450 gene of the present invention can be used also as a selection marker in introducing an additional gene.

The plant to be transformed in the present invention means any of an entire plant body, a plant organ (such as a leaf, a petal, a stem, a root, or a seed), a plant tissue (such as an epidermis, a phloem, a parenchyma, a xylem, or a vascular bundle), and a cultured plant cell. Examples of the plant to be used in the transformation include, but are not limited to, plants belonging to the Arabidopsis family, the Poaceae family, the Solanaceae family, and the Fabaceae family (see the following).
Arabidopsis family: Arabidopsis thaliana
Solanaceae family: tobacco (Nicotiana tabacum)
Poaceae family: corn (Zea mays) and rice (Oryza sativa)
Fabaceae family: soybean (Glycine max)

The above-described expression vector can be introduced into a plant by a usual transformation method, such as an electroporation method, an Agrobacterium method, a particle gun method, or a PEG method.

For example, when an electroporation method is employed, a treatment is performed with an electroporation device equipped with a pulse controller under conditions of a voltage of 500 to 1,600 V, 25 to 1,000 μF, and 20 to 30 msec to introduce the gene into a host.

Alternatively, when a particle gun method is employed, a plant body, plant organ, or plant tissue itself may be directly used, may be used after preparing a section, or may be used with a protoplast prepared. A sample thus prepared can be treated with a gene transfer device (such as PDS-1000/He available from Bio-Rad Laboratories, Inc.). Treatment conditions are varied depending on a plant or a sample, and the treatment is usually performed at a pressure of about 1,000 to 1,800 psi at a distance of about 5 to 6 cm.

Besides, the cytochrome P450 gene of the present invention can be introduced into a plant body by utilizing a plant virus as a vector. An example of the usable plant virus includes cauliflower mosaic virus. Specifically, a recombinant is first prepared by inserting a virus genome into an E. coli-derived vector or the like, and then, the cytochrome P450 gene of the present invention is inserted into the virus genome. The virus genome thus modified is cleaved from the recombinant with a restriction enzyme, and inoculated in a plant host, and thus, the cytochrome P450 gene of the present invention can be introduced into the plant host.

In a method utilizing Agrobacterium Ti plasmid, the cytochrome P450 gene of the present invention is introduced into a plant host by utilizing the following property: in a plant infected with a bacterium belonging to the genus Agrobacterium, part of its plasmid DNA is transferred into the plant genome. Among bacteria belonging to the genus Agrobacterium, Agrobacterium tumefaciens forms a tumor designated as crown gall through infection of a plant, and Agrobacterium rhizogenes generates a capillary root through infection of a plant. This is because a region designated as T-DNA region (transferred DNA) on a plasmid present in various bacteria designated as a Ti plasmid or Ri plasmid is transferred into a plant through infection to be integrated into a genome of the plant.

If a DNA desired to be integrated into a plant genome has been inserted into a T-DNA region on a Ti or Ri plasmid, the target DNA can be integrated into the plant genome when a bacterium belonging to the genus Agrobacterium infects the plant host.

A tumor tissue, a shoot, a capillary root, or the like resulting from the transformation can be directly used in cell culture, tissue culture, or organ culture, and can be regenerated into a plant body by a conventionally known plant tissue culture method with administration of an appropriate concentration of a plant hormone (such as auxin, cytokinin, gibberellin, abscisic acid, ethylene, or brassinolide) or the like.

A transformant obtained by introducing the cytochrome P450 gene of the present invention can be used for screening a compound usable as a novel herbicide for plants. Specifically, a candidate substance is contacted with a transformant obtained by introducing the cytochrome P450 gene of the present invention. Besides, the same candidate substance is contacted also with a cell into which the cytochrome P450 gene of the present invention has not been introduced (preferably, a host cell from which the transformant is obtained). Then, a compound with which the transformant grows but the cell not having the cytochrome P450 gene of the present invention introduced dies is selected. It can be concluded that the selected compound is a compound usable as a herbicide detoxified/metabolized by the cytochrome P450 gene of the present invention.

The screened compound is not toxic to a plant having the cytochrome P450 gene of the present invention, but is toxic to a plant not having the cytochrome P450 gene. Therefore, the screened compound can be used as a herbicide in selectively growing a plant having the cytochrome P450 gene of the present invention.

A transformant can be obtained by introducing the expression vector of the present invention not only into the plant host described above but also into bacteria belonging to the genus Escherichia such as E. coli, the genus Bacillus such as Bacillus subtilis, or the genus Pseudomonas such as Pseudomonas putida, yeasts such as Saccharomyces cerevisiae and Schizosaccharomyces pombe, animal cells such as COS cell and CHO cell, or insect cells such as Sf9. When a bacterium such as E. coli or yeast is used as a host, the expression vector of the present invention is preferably autonomously replicable in the bacterium, and at the same time, constructed to include the cytochrome P450 gene of the present invention, a ribosome binding sequence, a target gene, and a transcription termination sequence. Besides, a gene controlling the cytochrome P450 gene of the present invention may be included.

A method for introducing a recombinant vector into a bacterium is not especially limited as long as it is a method for introducing a DNA into a bacterium. Examples include a method using a calcium ion, and an electroporation method.

When yeast is used as the host, for example, Saccharomyces cerevisiae and Schizosaccharomyces pombe are used. A method for introducing a recombinant vector into yeast is not especially limited as long as it is a method for introducing a DNA into yeast, and examples include an electroporation method, a spheroplast method, and a lithium acetate method.

When an animal cell is used as the host, monkey cell COS-7, Vero, Chinese hamster ovarian cell (CHO cell), mouse L cell and the like are used. Examples of a method for introducing a recombinant vector into an animal cell include an electroporation method, a calcium phosphate method, and a lipofection method.

When an insect cell is used as the host, Sf9 cell and the like are used. Examples of a method for introducing a recombinant vector into an insect cell include a calcium phosphate method, a lipofection method, and an electroporation method.

It can be confirmed whether or not the gene has been integrated into the host by a PCR method, Southern hybridization method, Northern hybridization method, or the like. For example, a DNA is prepared from the transformant, and a DNA specific primer is designed to perform PCR. The PCR is performed under the same conditions as those employed for preparing the plasmid. Thereafter, an amplification product is subjected to agarose gel electrophoresis, polyacrylamide gel electrophoresis, capillary electrophoresis, or the like, the resultant is dyed with ethidium bromide, SYBR Green solution, or the like to detect the amplification product as one band, and thus, the transformation is confirmed. Alternatively, the amplification product can be detected by performing PCR using a primer precedently labeled with a fluorescent dye or the like. Further alternatively, a method in which the amplification product is bound to a solid phase such as a microplate to confirm the amplification product with a fluorescent or enzymatic reaction, or the like may be employed.

### 4. Production of Plant

In the present invention, a transformed plant body can be regenerated from the transformed plant cell or the like. As a regeneration method, a method in which transformed cells in a callus state are each transferred to media varied in the type and the concentration of hormones to be cultured therein, and adventitious embryos are thus formed to obtain a complete plant body is employed. Examples of the medium used include LS medium and MS medium.

A “method for producing a plant body” of the present invention comprises steps of obtaining a transformed plant cell by introducing, into a host cell, a plant expression vector comprising the cytochrome P450 gene of the present invention inserted thereinto, regenerating a transformed plant body from the transformed plant cell, obtaining a plant seed from the transformed plant obtained, and producing a plant body from the plant seed.

In order to obtain a plant seed from the transformed plant body, for example, the transformed plant body is collected from a rooting medium, transplanted in a pot holding soil containing water, and grown at a constant temperature to grow a flower, and ultimately to produce a seed. Besides, in order to produce a plant body from a seed, for example, when a seed grown on a transformed plant matures, the seed is isolated to be seeded in soil containing water, and grown at a constant temperature and constant brightness to produce a plant body. A plant thus produced expresses the cytochrome P450 gene of the present invention, and hence is resistant to the large number of drugs having different acting properties described above. Here, the term “being resistant to drugs” has the same meaning as being resistant to the drugs with a statistically significant difference as compared with before introducing the cytochrome P450 gene of the present invention. Resistance to a drug can be determined based on a mortality rate of a plant body, a growth inhibition rate of a stem and leaf portion, a root portion and the like, or the like obtained through contact with the drug at a prescribed concentration.

### 5. Method for Controlling Noxious Weed

A transgenic plant obtained by introducing the P450 gene of the present invention into a plant is resistant to a large number of drugs having different acting properties as described above. Therefore, when the transgenic plant obtained by introducing the P450 gene of the present invention into a plant is cultivated in a field, a drug selected from the large number of drugs having different acting properties described above can be used as a herbicide to wither weed excluding the transgenic plant.

As described so far, a transgenic plant obtained by introducing the P450 gene of the present invention into a plant can be cultivated with weed easily removed because a drug for removing weed in a field can be selected from a wide range.

### EXAMPLES

Now, the present invention will be described in more detail with reference to examples, and it is noted that the technical scope of the present invention is not limited to the following examples.

### Example 1

### 1. Search for Novel Wheat P450 Metabolizing Herbicide

### 1) Search and Isolation of Wheat P450 Gene Metabolizing Fenquinotrione

It is currently known that CYP81A6 of a rice plant has metabolic activity against three drugs having different mechanisms of action, that is, an ALS inhibitor, a photosystem II inhibitor, and an HPPD inhibitor in HRAC classifications. Among these, fenquinotrione, that is, an HPPD inhibitor for paddy rice, is metabolically degraded through demethylation with CYP81A6, and hence has been revealed to have high safety for a rice plant. This drug also has high safety for wheat, and a main metabolite of this drug in wheat is a demethylated product as in a rice plant, and hence, it was suggested that a P450 having a similar function to CYP81A6 is present in wheat.

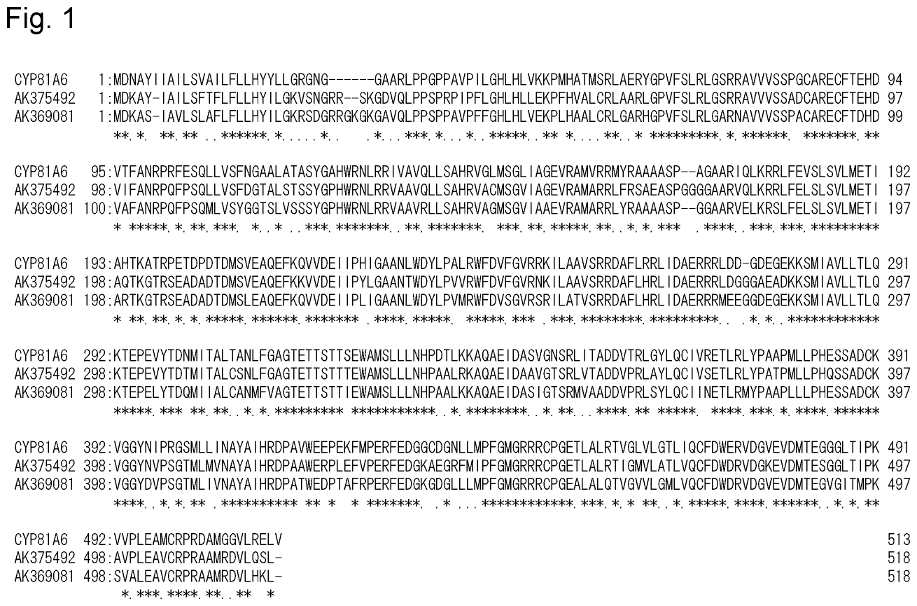

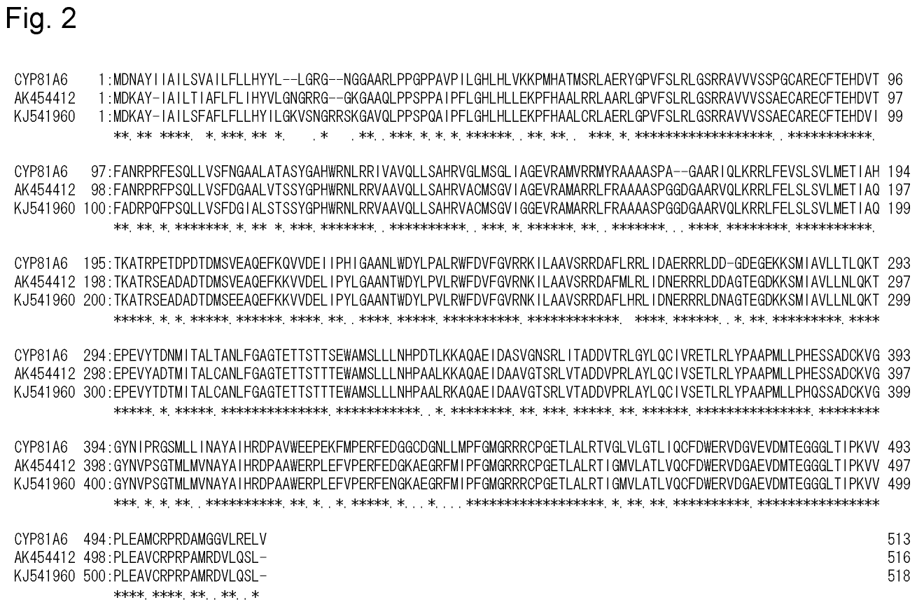

Since CYP71C6v1 known as a P450 for metabolizing a herbicide in wheat was, however, not highly homologous to CYP81A6, it was presumed that metabolically degrading activity against fenquinotrione is caused by an unknown P450 different from CYP71C6v1. Therefore, genes similar to CYP81A6 in wheat were searched for. With the ORF sequence of CYP81A6 used as a query, blastn search was performed on GenBank. As a result, two barley-derived P450s (accession No. AK375492 (80%) and AK369081 (80%)) and one wheat-derived P450 (accession No. AK454412 (clone ID: tplb0005j01) (81%)) were hit. Besides, with the ORF sequence of AK454412 used as a query, blastn search was performed on GenBank, and as a result, another wheat-derived P450 of Accession No. KJ541960 (80%) was hit (it is noted that each parenthesized numeral indicates identity to CDS sequence of CY81A6 gene). FIG. 1 illustrates multiple alignment of CYP81A6, AK375492, and AK369081, and FIG. 2 illustrates multiple alignment of CYP81A6, AK454412, and KJ541960.

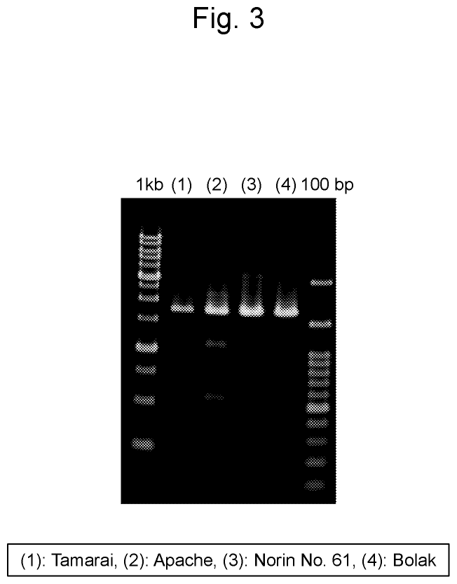

In order to verify fenquinotrione metabolic activity in vitro of AK454412 gene, that is, the wheat-derived putative P450 gene hit, this gene was cloned, and it was verified whether or not it is present in various breeds of wheat. The breeds of wheat tested were Norin No. 61 produced in Japan, Bolak produced in Australia, Apache produced in Europe, and Tamarai as durum wheat. A full length of the CDS region sandwiching the UTR region was amplified via PCR with a cDNA of each of these four breeds used as a template and using a primer set of AK454412 5′-UTR (Fd) and AK454412 3′-UTR (Rv). As a result, a band (1,791 bp) was confirmed in a predicted position in all of the breeds, and hence it was suggested that this gene may be present regardless of breed (FIG. 3). Subsequently, to clone this gene, PCR was performed again with a DNA fragment amplified with a genome of the Apache wheat used as a template, and using a primer set of AK454412 IF (Fd) and AK454412 IF (Rv), and thus, the CDS sequence of the AK454412 gene was amplified. This DNA fragment and pET-22b(+) vector, having been treated with restriction enzymes NdeI and XhoI, were linked to each other with In-Fusion HD Cloning Kit (TaKaRa) to produce pET-22b-AK454412. The thus obtained construct was subjected to sequence analysis, resulting in finding that, as compared with the sequence described in GenBank, the 453rd C was mutated to T, and the 1,137th T was mutated to C, both of which were mutations not involving amino acid mutation.

The sequences of the primers used were as follows

AK454412 5′-UTR (Fd):
(SEQ ID No. 17)
5′-CAACCTGAGACCTCAAGTGTCAC-3′
AK454412 3′-UTR (Rv):
(SEQ ID No. 18)
5′-GTACTACCTGGATCCACGAGC-3′
AK454412 IF (Fd):
(SEQ ID No. 19)
5′-AAGGAGATATACATATGGATAAGGCGTACATTGCC-3′
AK454412 IF (Rv):
(SEQ ID No. 20)
5′-GTGGTGGTGCTCGAGTCAGAGGCTCTGAAGCACGT-3′

### 2) Search and Isolation of Wheat-Derived P450 Reductase

In general, for monooxygenase reaction of P450, two-electron reduction, and accompanying proton transfer are indispensable. An enzyme catalyzing this reaction is P450 reductase (CPR), and wheat-derived CPR is necessary for AK454412, presumed as one of wheat P450s, to function. Therefore, it was decided to search and clone wheat-derived CPR. First, with Os09g0558900, that is, rice P450 reductase (CPR), used as a query, Blast search was performed on GenBank. As a result, two genes of accession No. AK333516 (derived from Chinese Spring, SEQ ID NO: 21) and AJ303373 (derived from Darius, SEQ ID NO: 22) were found, and since the CDS sequences of these genes were substantially the same (with the 1,665th A replaced with G (amino acid: I555V) when seen in AJ303373), these genes were regarded as the same gene, and hence, it was here decided to perform cloning from AJ303373 (hereinafter referred to as TaCPR).

First, in the same manner as in the search for a wheat P450, with a cDNA of each of the four breeds, Norin No. 61, Bolak, Apache, and Tamarai, used as a template, a full length of the CDS region sandwiching the UTR region was amplified via PCR using a primer set of CPR_AJ303373 UTR (Fd) and CPR UTR (Rv). As a result, a band (2,397 bp) in a predicted position was confirmed in all of the breeds tested here, and hence it was suggested that this gene may be present regardless of breed. Subsequently, to clone this gene, PCR was performed again with a DNA fragment amplified with a genome of the Apache wheat used as a template, and using a primer set of TaCPR (AJ303373) IF (Fd) and TaCPR IF (Rv), and thus, the CDS sequence of the AJ303373 gene was amplified. This DNA fragment and pACYC Duet-2 vector (Novagen), having been treated with restriction enzymes NdeI/XhoI, were linked to each other with In-Fusion HD Cloning Kit (TaKaRa) to produce pACYC-TaCPR. The thus obtained construct was subjected to sequence analysis, resulting in finding that the sequence of the cloned AJ303373 gene completely matched the sequence described in database.

The sequences of the primers used were as follows:

CP_RAJ303373_UTR (Fd):
(SEQ ID No. 23)
5′-CACGCGTCCGATCGAACCAAC-3′
CPR_UTR (Rv):
(SEQ ID No. 24)
5′-CGATGAATCGTCATCCTCTGTTCCAC-3′
TaCPR(AJ303373) IF (Fd):
(SEQ ID No. 25)
5′-AAGGAGATATACATATGGACTCCGCCGCCGCGGGG-3′
TaCPR IF (Rv):
(SEQ ID No. 26)
5′-CTTTACCAGACTCGATTACCAGACGTCTCTCAGGT-3′

3) Preparation of AK454412 Construct for Expressing E. coli

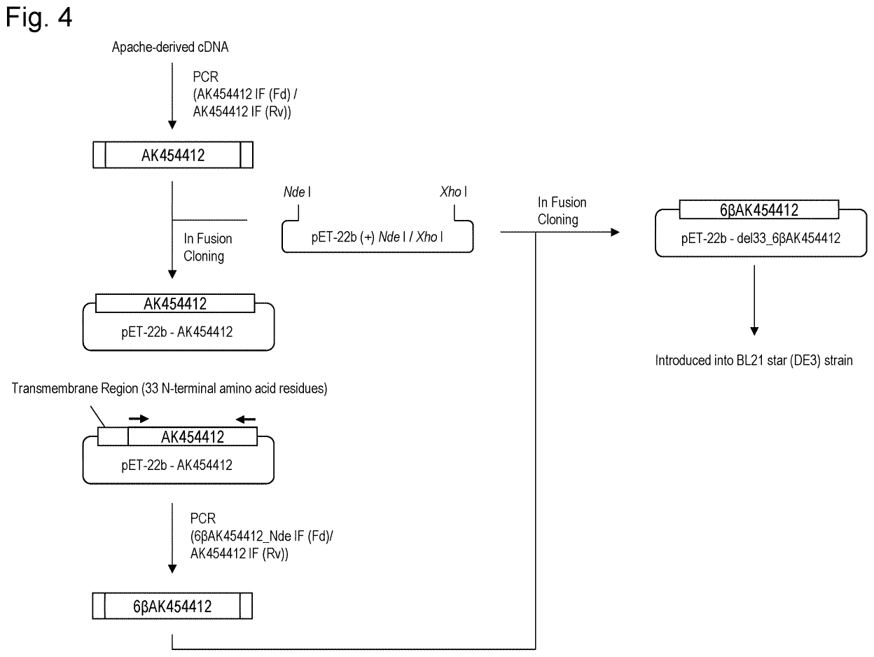

A P450 of a eukaryote usually has a transmembrane region (membrane anchoring region) at the N-terminal, and it is known that expression possibility is increased in expression of E. coli by removing this region, and adding, to the N-terminal, Barnes sequence (MALLLAV) or rabbit 6p-hydroxylase-derived sequence (6p sequence: MAKKTSS). Therefore, with the above-described pET-22b-AK454412 plasmid used as a template, PCR was performed using a primer set of 6β AK454412_Nde IF (Fd) and AK454412 IF (Rv) to obtain a DNA fragment in which 15 bases complementary to the vector side terminal were further added to both terminals of a nucleotide sequence encoding a sequence obtained by deleting 33 N-terminal amino acid residues of AK454412, and adding 7 amino acid residues (6p sequence: MAKKTSS). This DNA fragment and pET-22b(+), having been treated with NdeIXhoI, were linked to each other with In-Fusion HD Cloning Kit (Clontech) to construct pET-22b-del33_6β AK454412. The prepared construct was introduced into E. coli BL21 star (DE3) strain (FIG. 4).

The sequences of the primers used were as follows:

6βAK454412_NdeIF (Fd):
(SEQ ID No. 27)
5′-AAGGAGATATACATATGGCTAAGAAGACTTCTTCT-3′
AK454412 IF (Rv):
(SEQ ID No. 28)
5′-GTGGTGGTGCTCGAGTCAGAGGCTCTGAAGCACGT-3′

4) Preparation of AJ303373 Construct for Expressing E. coli

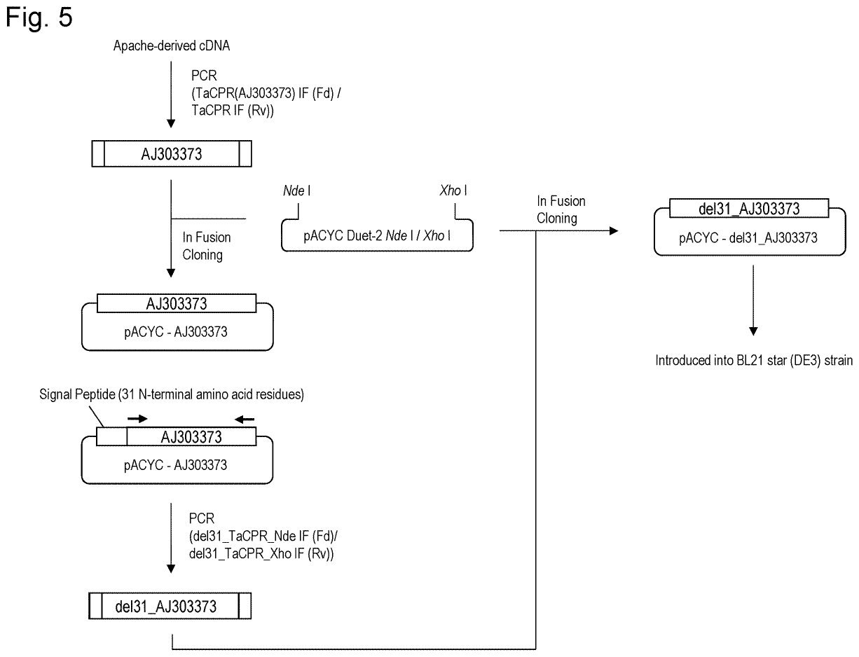

CPR has chloroplast transfer signal peptide at the N-terminal, and heterologous expression can be caused in E. coli by removing this sequence. Therefore, with the above-described pACYC-TaCPR plasmid used as a template, PCR was performed using a primer set of del31_TaCPR_Nde IF (Fd) and del31_TaCPR_Xho IF (Rv) to obtain a DNA fragment in which 15 bases complementary to the vector side terminal were further added to both terminals of a nucleotide sequence encoding a sequence obtained by deleting 31 N-terminal amino acid residues of AJ303374. This DNA fragment and pACYC Duet-2, having been treated with NdeI/XhoI, were linked to each other with In-Fusion Cloning HD (Clontech) to construct pACYC-del31_TaCPR. The prepared construct was introduced into E. coli BL21 star (DE3) strain (FIG. 5).

The sequences of the rimers used were as follows:

del31_TaCPR_Nde IF (Fd):
(SEQ ID No. 29)
5′-AAGGAGATATACATATGGACCAGAACCGCCGCCTG-3′
del31_TaCPR_Xho IF (Rv):
(SEQ ID No. 30)
5′-TTTACCAGACTCGAGTTACCAGACGTCTCTCAGGT-3′

5) Protein Co-Expression of AK454412 and AJ303373 in E. coli Expression System

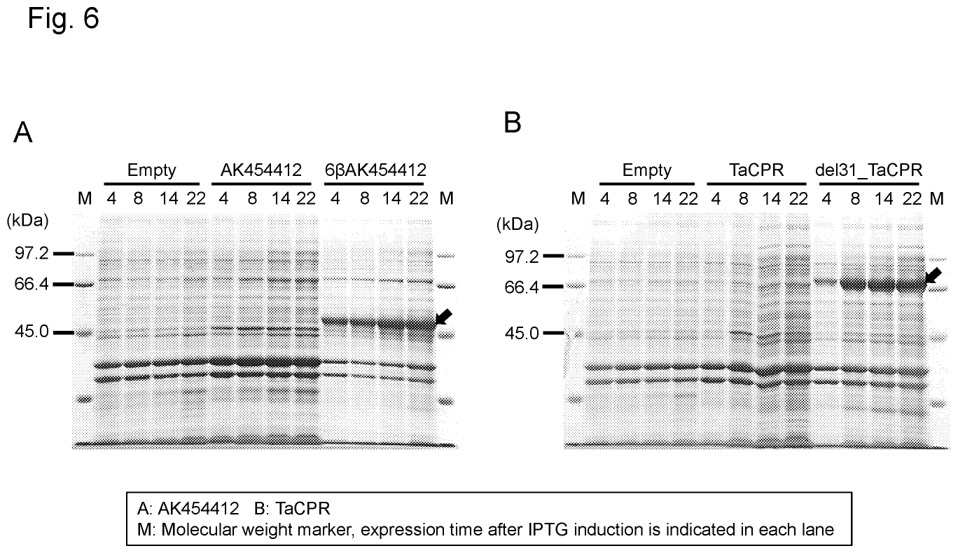

A single colony of the recombinant E. coli into which each of the above-described two expression constructs had been introduced was inoculated in 3 ml of LB liquid medium, followed by culturing at 37° C. and 200 rpm overnight. 2.5 mL of the resultant culture fluid was added to 250 mL of TB medium (containing 100 ppm carbenicillin and 50 ppm chloramphenicol) held in a 1 L Erlenmeyer flask, followed by culturing at 37° C. and 200 rpm until an OD₆₀₀ of about 0.5 to 0.6 was obtained. After the resultant flask was cooled with ice, IPTG and aminolaevulinic acid were added thereto to final concentrations of 1 mM and 0.5 mM, respectively, followed by culturing at 20° C. and 110 rpm. 22 hours after the IPTG induction, the resultant culture fluid was centrifuged (4° C., 15,000 rpm, 1 min). A bacterial cell in the obtained 500 mL of culture fluid was suspended in 35 mL of P450 Buffer [50 mM potassium phosphate buffer (pH 7.3), 20% glycerol, 1 mM EDTA, and 1 mM DTT] to be sonicated, followed by centrifugation at 4° C. and at 6,000×g for 10 minutes. The supernatant was centrifuged at 4° C. and at 100,000×g for 1 hour, and a solution obtained by adding P450 Buffer by 1 mL to the obtained precipitate and homogenizing the resultant with a Teflon homogenizer was used as a co-expression crude enzyme solution for AK454412 and TaCPR. This crude enzyme solution was used to examine protein expression by SDS-PAGE (CBB dyeing), and bands of AK454412 and TaCPR were confirmed in the vicinity of 54 kDa and 75 kDa, respectively, and thus, these enzymes were successfully expressed. Results of the SDS-PAGE are illustrated in FIG. 6. FIG. 6(A) illustrates a result (with an arrow) of the expression of AK454412 (6p AK454412) in which 33 N-terminal amino acid residues were removed and the 6β sequence was added, and FIG. 6(B) illustrates a result (with an arrow) of the expression of TaCPR (del31_TaCPR) in which 31 N-terminal amino acid residues were removed.

### 6) In Vitro Metabolism Test Using Co-Expression Crude Enzyme of AK454412 and TaCPR

It was verified by in vitro metabolism test whether or not a novel wheat-derived P450 of AK454412 is involved in herbicide metabolism. As a compound having high safety against wheat, fenquinotrione was used. A reaction solution had a composition of 50 mM potassium phosphate buffer (pH 7.3), 1 mg of the crude enzyme, 1 mM β-NADPH, and 10 μM fenquinotrione, and the reaction was performed at 30° C. for 1 hour, and then stopped by vortex. A solution after completing the enzymatic reaction was centrifuged at 15,000 rpm for 1 minute, and the obtained supernatant was filtered through a 0.45 μm filter and then tested by LC/MS. LC/MS conditions were as follows:

### <HPLC Conditions>

apparatus: Prominence UFLC (Shimadzu Corporation)
detector: UV 254 nm
temperature: 35° C.
flow rate: 0.3 mL/min
column: TSKgel ODS-80 Ts (0.5 μm, 2.0 mm×150 mm, TOSOH)
mobile phase: acetonitrile (containing 0.5% acetic acid)/water (containing 0.5% acetic acid)=20/80 (5 min hold)→(15 min)→80/20 (10 min hold)→(4 min)→20/80 (6 min hold)

### <MS Conditions>

apparatus: Triple Quad™ 4500 (SCIEX)
interface: ESI
scan mode: Q1 Scan, Product ion scan m/z 140-700
polarity: positive
ion spray voltage: 4,500
source temperature: 400
curtain gas: 40
ion source gas 1: 50
ion source gas 2: 50
declustering potential: 20
entrance potential: 4.5

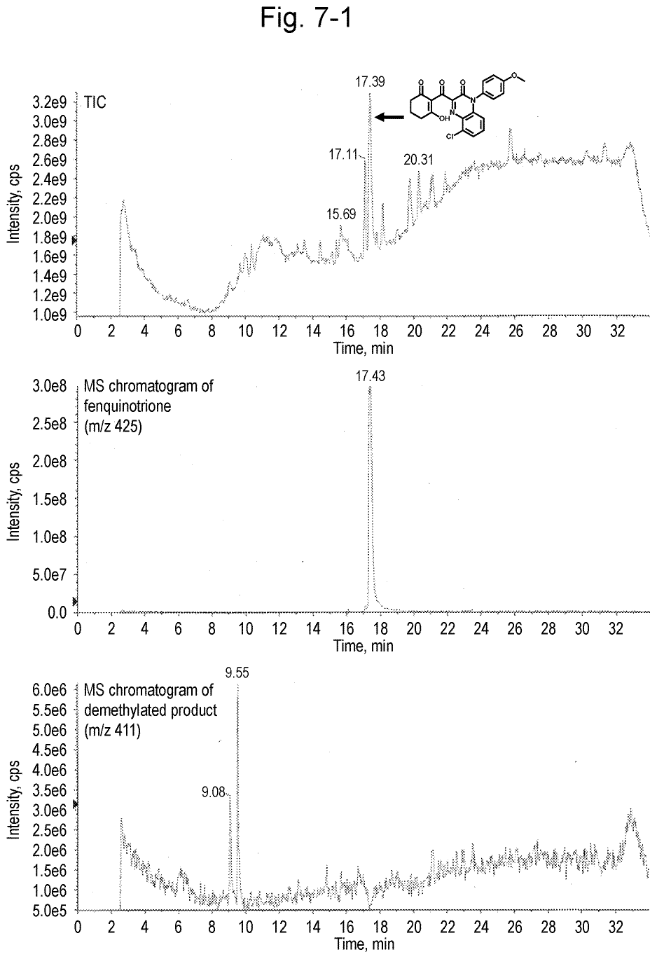

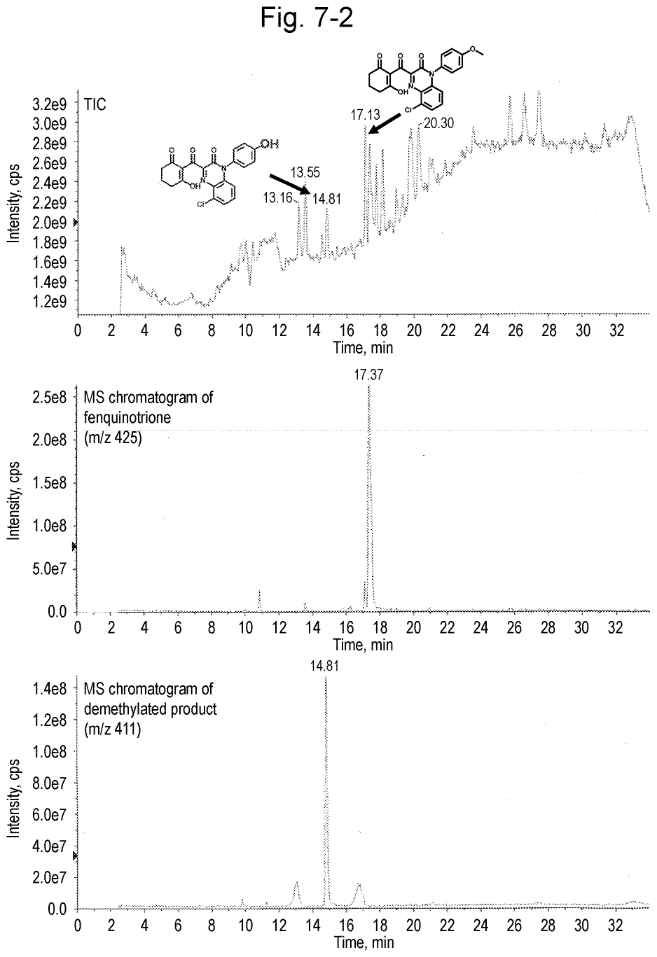

Results of the LC/MS analysis are illustrated in FIGS. 7-1 and 7-2. FIG. 7-1 illustrates a result of the LC/MS analysis performed on a negative control plot (empty vector expression crude enzyme reaction plot). FIG. 7-2 illustrates a result of LC/MS analysis performed on an AK454412-TaCPR co-expression crude enzyme reaction plot. As illustrated in FIG. 7-2, when the AK454412 and TaCPR co-expression crude enzyme solution was used, peaks were detected in the vicinity of RT of 17.4 and 14.8, and molecular weight of these peaks were m/z 425, and m/z 411, respectively. As a result of comparison with a preparation, it was revealed that the peak of m/z 425 corresponds to the parent compound of fenquinotrione, and the peak of m/z 411 corresponds to a demethylated product of fenquinotrione. This demethylated product is a main metabolite of fenquinotrione in a wheat body, and it has been known that this metabolite does not exhibit herbicidal activity. It was revealed, based on these, that AK454412 is a P450 that metabolically degrades fenquinotrione to the demethylated product to impart wheat selectivity.

### [Example 2] Functional Analysis of Novel Wheat-Derived P450, AK454412

1) Creation of Arabidopsis thaliana Having AK454412 Forced Expression

In order to examine the influence of the demethylation reaction of fenquinotrione verified in the in vitro test on a plant body, recombinant Arabidopsis thaliana having the AK454412 gene introduced thereinto was created to examine a difference in fenquinotrione sensitivity from wild type.

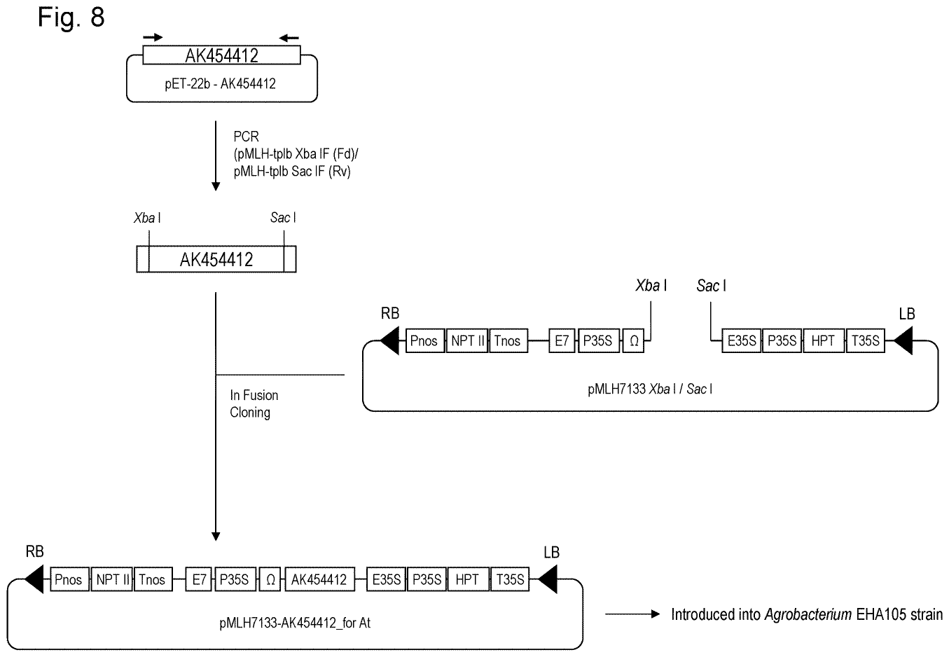

First, a binary vector, pMLH7133-AK454412_for At, for transforming Arabidopsis thaliana was produced. With the pET-22b-AK454412 produced in Example 1 used as a template, PCR was performed using a primer set of pMLH-tplb Xba IF (Fd) and pMLH-tplb Sac IF (Rv) to obtain a DNA fragment in which XbaI/SacI recognition sequence and 15 bases of In-Fusion recognition sequence were added to a terminal of full length CDS sequence of the AK454412 gene. The obtained DNA fragment and pMLH7133 vector, having been digested with the restriction enzymes XbaI/SacI, were linked to each other with In-Fusion HD Cloning Kit (Clontech) to produce a binary vector, pMLH7133-AK454412_for At, for transforming Arabidopsis thaliana (FIG. 8). The obtained construct was introduced into Agrobacterium EHA105 strain by an electroporation method. The transformation of Arabidopsis thaliana was performed by a floral dip method using this recombinant Agrobacterium.

The sequences of the primers used were as follows:

pMLH-tplb Xba IF (Fd):
(SEQ ID No. 31)
5′-TACAACTACATCTAGAATGGATAAGGCGTACATTG-3′
pMLH-tplb Sac IF (Rv):
(SEQ ID No. 32)
5′-GGGGAAATTCGAGCTCTCAGAGGCTCTGAAGCACG-3′

2) Fenquinotrione Sensitivity Test Using Arabidopsis thaliana Having AK454412 Forced Expression

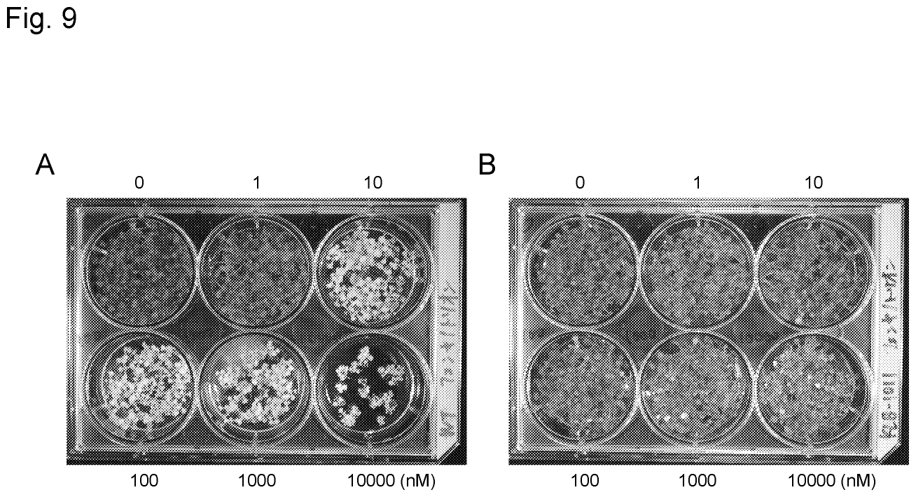

With a sensitivity difference from wild type Arabidopsis thaliana used as an index, it was verified whether or not the created transformant had resistance to fenquinotrione. In MS solid media in each of which fenquinotrione was added to a final concentration of 10,000, 1,000, 100, 10, or 1 nM, or 0.1% of DMSO was added as an untreated plot, sterilized seeds of Arabidopsis thaliana having AK454412 forced expression and wild type Arabidopsis thaliana were each seeded, and grown at 22° C. for about 10 days. The sensitivity to this drug was evaluated subjectively in accordance with the degree of chlorosis of acting symptom. The results are illustrated in FIG. 9. FIG. 9(A) illustrates results obtained in using the wild type Arabidopsis thaliana, and FIG. 9(B) illustrates results obtained in using the Arabidopsis thaliana having AK454412 forced expression. As illustrated in FIG. 9, 90% or more of the wild type Arabidopsis thaliana had chlorosis through the treatment with 10 nM fenquinotrione, but the chlorosis symptom was not observed in the Arabidopsis thaliana having AK454412 forced expression even with 10,000 nM fenquinotrione. This result revealed that the AK454412 gene is greatly involved in metabolic degradation of fenquinotrione, and imparts resistance to fenquinotrione to a plant when expressed therein.

### [Example 3] Test of Sensitivity to Various Drugs Using Arabidopsis thaliana Having AK454412 Forced Expression

It was revealed that the safety factor of fenquinotrione in wheat is metabolic degradation caused by AK454412. On the other hand, it has been shown that CYP81A6 metabolically degrades fenquinotrione in a rice plant, and it has been reported that it is involved in metabolic degradation of not only fenquinotrione but also sulfonylurea-based ALS inhibitors such as bentazone and bensulfuron-methyl (Gang Pan et al, Plant Mol Biol (2006) 61:933-943). This CYP81A6 and the wheat P450 of AK454412 are comparatively highly homologous to each other, and hence it was presumed that the AK454412 gene may be also involved in the metabolic degradation of these drugs similarly to CYP81A6.

Therefore, similar tests were performed on compounds having various acting properties described in HRAC including these sulfonylurea-based ALS inhibitors to search for a compound that can be metabolically degraded by AK454412. Specifically, 61 compounds arbitrarily selected from all of the 25 classifications of HRAC 2020 were subjected to sensitivity examination in Arabidopsis thaliana having AK454412 introduced thereinto and wild type. The sensitivity to each of the drugs was evaluated subjectively with respect to every acting symptom.

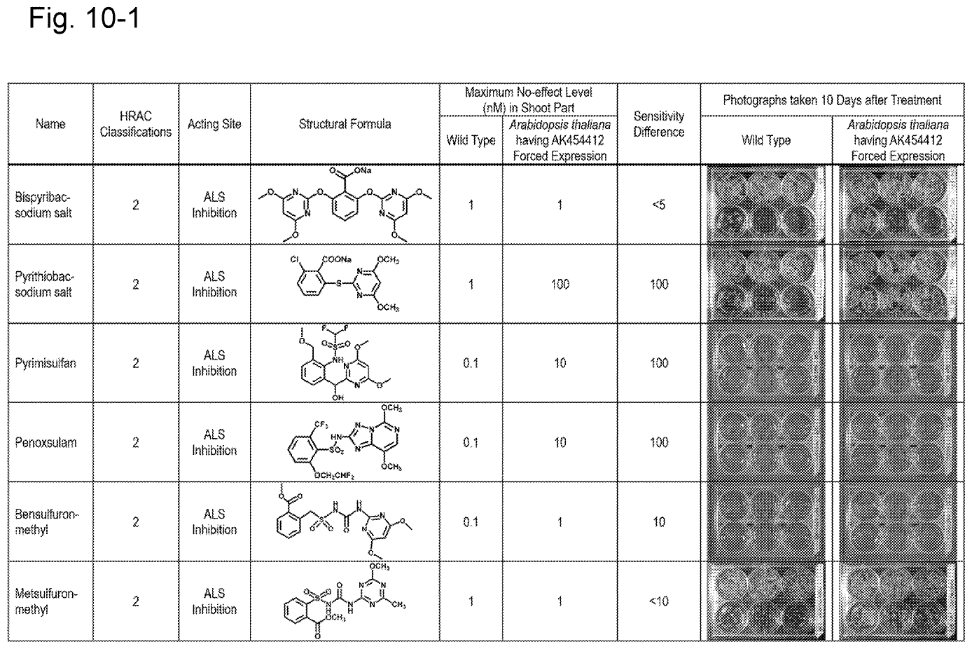

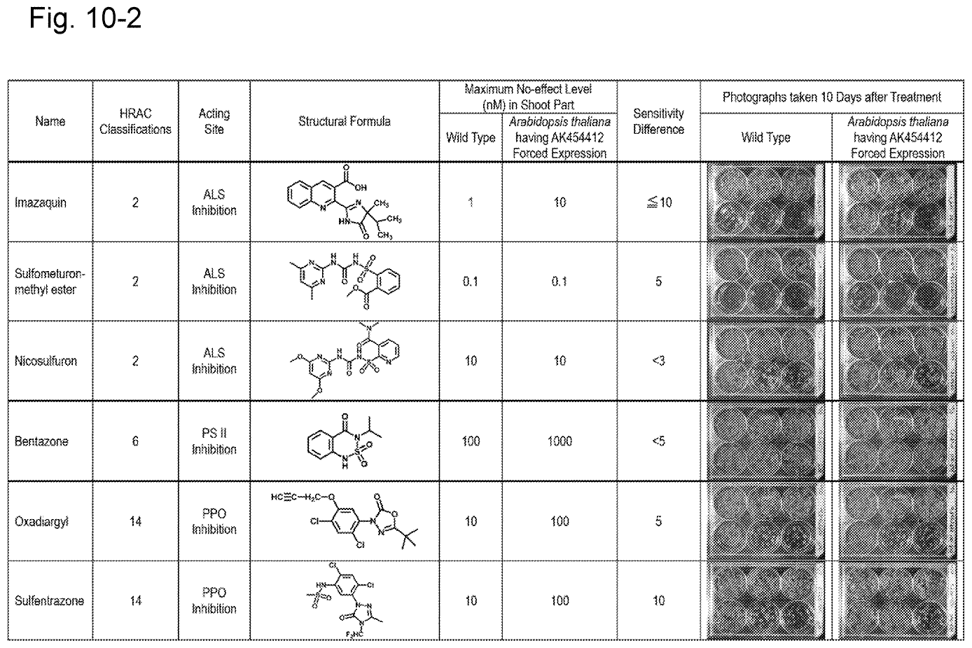

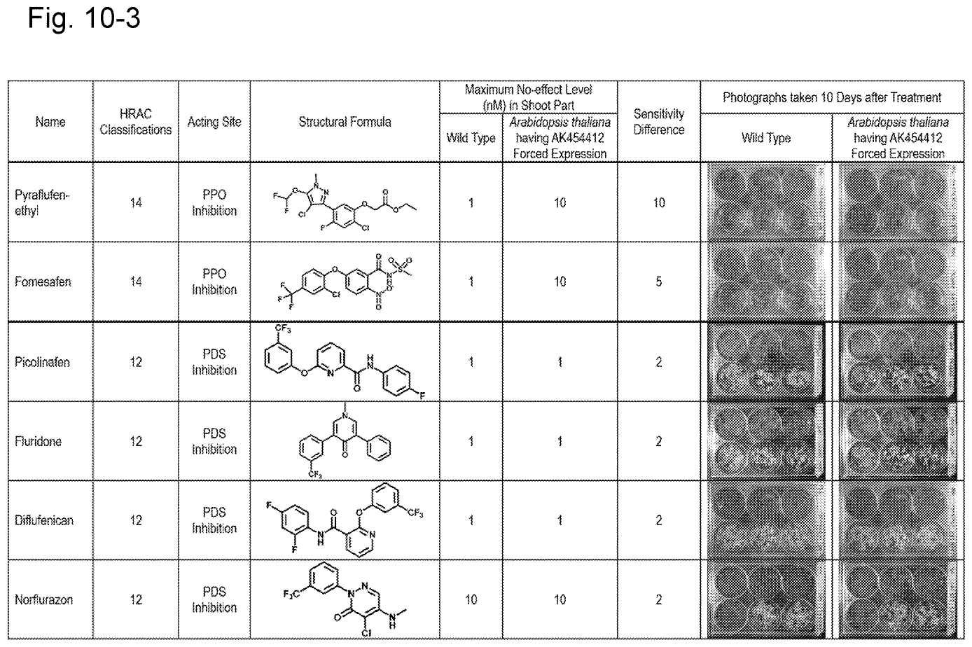

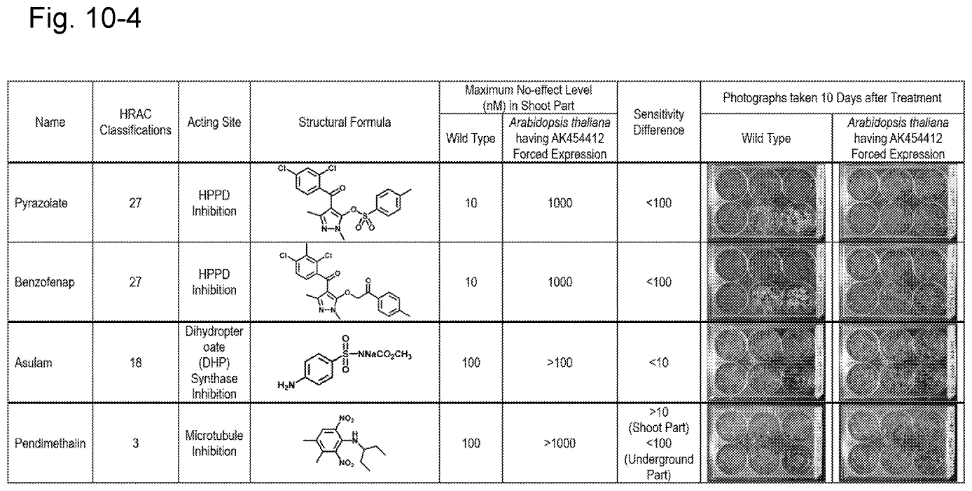

As a result, compounds found to have a difference in the sensitivity between the wild type and the recombinant Arabidopsis thaliana are illustrated in FIGS. 10-1 to 10-4. It is noted that concentrations of the drug in respective wells of a plate shown in each photograph in FIGS. 10-1 to 10-4 are 0, 0.1, and 1 nM in the stated order in the rightward direction in an upper portion, and 10, 100, and 1,000 nM in the stated order in the rightward direction in a lower portion. Besides, the compound found to have a sensitivity difference are listed below. Each parenthesized numeral indicates an approximate sensitivity difference evaluated subjectively based on a drug concentration exhibiting the effect.
HRAC code 2 (ALS inhibitors)
Bispyribac-sodium salt (<5), pyrithiobac-sodium salt (100), pyrimisulfan (100), penoxsulam (100), bensulfuron-methyl (10), metsulfuron-methyl (<10), imazaquin (<10), nicosulfuron (<10), sulfometuron-methyl (5)
HRAC code 6 (PS II inhibitor)
bentazone (<5)
HRAC code 14 (PPO inhibitors)
oxadiargyl (2), sulfentrazone (10), pyraflufen-ethyl (10), fomesafen (3)
HRAC code 12 (PDS inhibitors)
diflufenican (2), picolinafen (2), fluridone (2), norflurazon (2)
HRAC code 27 (HPPD inhibitors)
pyrazolate (<100), benzofenap (<100)
HRAC code 18 (DHP synthase inhibitor)
asulam (<10)
HRAC code 3 (microtubule assembly inhibitor)
pendimethalin (<100)

Based on these results, it was revealed that the compounds that can be metabolically degraded by AK454412 are 23 compounds among all the 62 compounds including fenquinotrione, and are the acetolactate synthase inhibitors classified as code 2 of HRAC classifications, the photosystem II inhibitor classified as code 6 of HRAC classifications, the protoporphyrinogen oxidase inhibitors classified as code 14 of HRAC classifications, the carotenoid biosynthesis inhibitors in the phytoene desaturase (PDS) classified as code 12 of HRAC classifications, the 4-hydroxyphenylpyruvate dioxygenase inhibitors classified as code 27 of HRAC classifications, the dihydropteroate synthase inhibitor classified as code 18 of HRAC classifications, and the microtubule assembly inhibitor classified as code 3 of HRAC classifications.

On the other hand, it is only bentazone, sulfonylurea-based ALS inhibitors such as bensulfuron-methyl, fenquinotrione, pyrimisulfan, and pyriminobac-methyl (three acting properties in HRAC classifications) that have been reported so far as compounds against which CYP81A6 exhibits metabolic reactivity. This also revealed that the AK454412 gene is highly homologous to the CYP81A6 gene, but encodes a P450 largely different in substrate recognition. In particular, it was revealed that the wheat-derived AK454412 gene can metabolically degrade a larger number of drugs having different acting properties as compared with the rice-derived homologous gene CYP81A6 and the conventionally known P450 genes.

### [Example 4] Verification of Conservation of AK454412 Gene in Respective Wheat Breeds

As described above, it was revealed that the AK454412 gene of a wheat-derived novel P450 can detoxify/metabolize the largest number of drugs among all P450s of plants found so far. In the above-described examples, the AK454412 gene isolated from Apache, which is a breed of bread wheat, was used for the examination, and in order to verify whether or not this gene functions regardless of wheat breed, conservation of this gene in various wheat breeds was examined.

Specifically, the verification was conducted by performing gene sequence analysis and gene expression level analysis on 13 breeds of bread wheat (Yumekaori, Hanamanten, Yumeseiki, Norin No. 61, Kitahonami, Kinuhikari, Shunyou, Bolak, Sentinel, EGA Wedgetail, Gatalina, Yopti, and Apache), and 5 breeds of durum wheat (Orlo, Miradoux, Argeles, Tamarai, and Jandaroi).

A method for the gene sequence analysis will be described below. A wheat seedling cultivated in Hoagland's No. 2 (Sigma-Aldrich) solid medium for about 1 week was frozen crushed with liquid nitrogen, and a total RNA was extracted therefrom with RNeasy Plant Mini Kit (Qiagen). The obtained total RNA was subjected to DNase I treatment with Deoxyribonuclease RT Grade for Heat Stop (NIPPON GENE CO., LTD.), and then a cDNA was synthesized with Transcriptor First Strand cDNA Synthesis Kit (Roche). For a reverse transcription reaction, Oligo dT Primer and Random Hexamer Primer were both used. With the produced cDNA of each wheat breed used as a template, PCR was performed using a primer set of AK454412 5′-UTR (Fd) and AK454412 3′-UTR (Rv) to amplify a region from 5′-UTR to 3′-UTR of the gene. The resultant DNA fragments were purified with GFX PCR DNA and Gel Band Purification Kit (GE Healthcare Science), and subjected to sequence analysis using various primers.

As a result of the sequence analysis performed on the 18 breeds of bread wheat and durum wheat, Norin No. 61, Kinuhikari, Sentinel, and EGA Wedgetail of the bread wheat breeds had a gene sequence completely matching the nucleotide sequence of the AK454412 gene described in GenBank. Besides, Miradoux, Argeles, Tamarai, and Jandaroi of the durum wheat breeds had a gene sequence obtained by mutating the 1,137th T to C (C1137T) in the nucleotide sequence of the AK454412 gene. In addition, Yumekaori, Hanamanten, Yumeseiki, Kitahonami, Shunyou, Bolak, Gatalina, Yipti, and Apache of the bread wheat breeds had a gene sequence obtained by mutating the 453rd C to T (C453T) and the 1,137th T to C (T1137C) in the nucleotide sequence of the AK454412 gene. It is noted that these two mutations are silent mutations, and did not affect the amino acid sequences. Besides, Orlo of the durum wheat breed had a gene sequence including C453T, G1114A, T1137C, and C1179T mutations in the nucleotide sequence of the AK454412 gene. Among these mutations, the G1114A (V372M) mutation alone was a mutation involving amino acid mutation. It was found, based on these results, that almost all the breeds tested in the present example have the same gene as or functionally equivalent gene to the AK454412 gene.

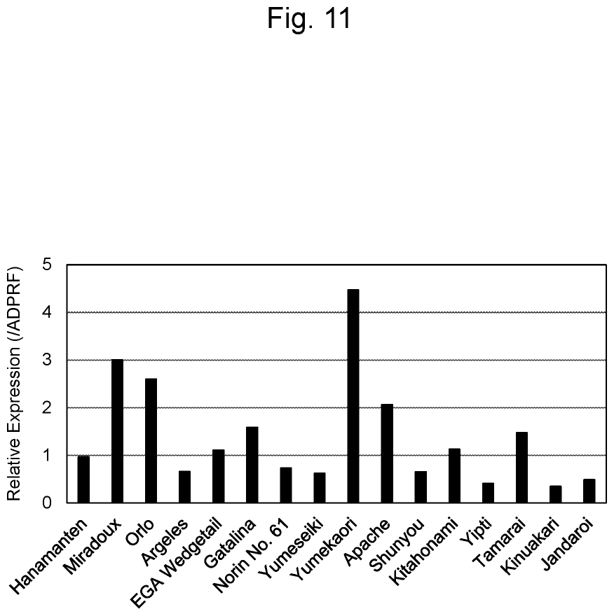

Subsequently, the expression analysis was performed as follows. Real time PCR was performed with the cDNA of each wheat breed produced as described above used a template. In order to prepare a reaction system of the real time PCR, GoTaq qPCR Master Mix (Promega Corporation) was used, and Thermal Cycler Dice Real Time System TP800 (TaKaRa) was used as a thermal cycler. As reaction conditions for the PCR, initial denaturation was performed at 95° C. for 30 seconds, denaturation was performed at 95° C. for 5 seconds, and annealing/elongation was performed at 60° C. for 30 seconds (45 cycles). As a reference gene, ADP-Ribosylation Factor gene (accession No. AB050957) was used. As an analysis method for a Ct value, a Crossing Point method was employed, and relative quantification was calculated by a calibration curve method. The results are illustrated in FIG. 11. As illustrated in FIG. 11, the gene expression level was about 0.5 to 4.5 in terms of a ratio to reference gene expression in all the breeds, and thus, it was found that they had comparatively high expression levels equivalent to or higher than that of the reference gene.

It was thus reveled that the AK454412 gene is conserved in various wheat breeds, and functions therein.

The sequences of the primers used were as follows:

(SEQ ID No. 33)
ADP-RF (Fd); 5′-GCTCTCCAACAACATTGCCAAC-3′
(SEQ ID No. 34)
ADP-RF (Rv); 5′-GCTTCTGCCTGTCACATACGC-3′
(SEQ ID No. 35)
AK454412 RT (Fd); 5′-CTCTGCGCGAATTTATTTGG-3′
(SEQ ID No. 36)
AK454412 RT (Rv); 5′-TCACGATGCACTGCAGGTAG-3′

### [Example 5] Functional Analysis of KJ541960, Homologous Gene to AK454412

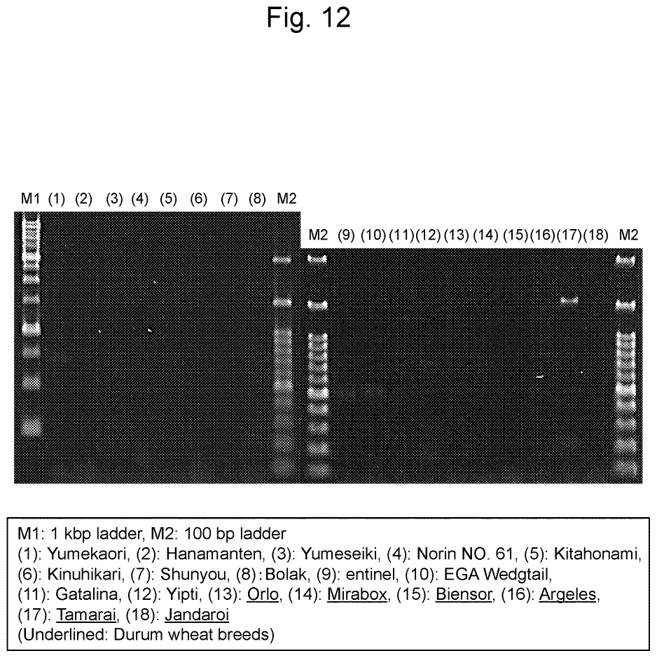

As described in Example 1, when blast search was performed on GenBank with the AK454412 gene used as a query, Accession No. KJ541960 (having homology of 96% identity in an amino acid sequence excluding a transmembrane region to the AK454412 gene) was hit as the wheat-derived putative P450 gene. Because of high homology to the AK454412 gene, it was also verified whether or not the KJ541960 gene has the similar function to the AK454412 gene. With a cDNA of each of the 18 wheat breeds used as a template, PCR was performed using a primer set of KJ541960 5′-UTR (Fd) and KJ541960 3′-UTR (Rv) to amplify a region from 5′-UTR to 3′-UTR of KJ541960. As a result, a band was found in the vicinity of a predicted position in only one breed (Tamarai) (FIG. 12). In order to check whether or not this DNA fragment matches the target gene, this DNA fragment was subjected to sequence analysis, and it was revealed that this was not the target gene. Based on these results, it was presumed that this gene may not be present in the wheat breeds tested here.

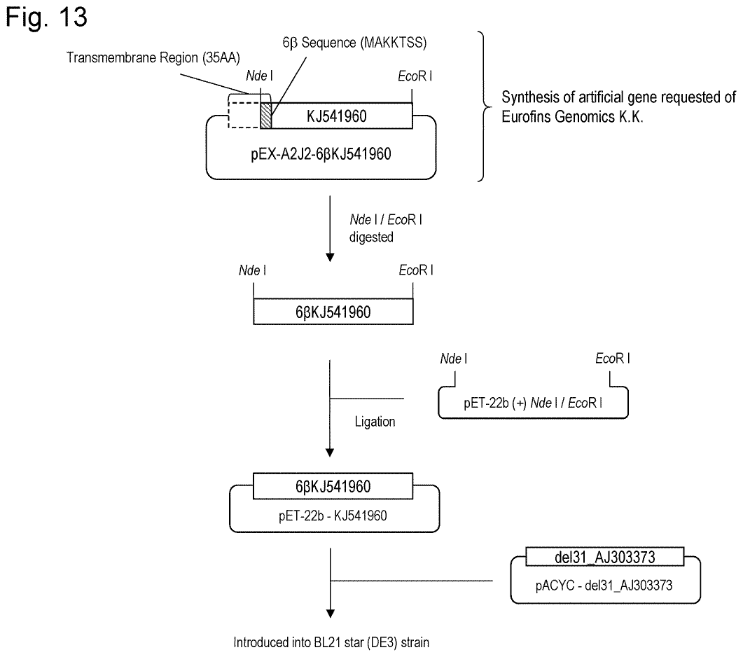

Therefore, synthesis of an artificial gene of the KJ541960 gene was requested of Eurofins Genomics K.K. Here, NdeI and EcoRI sites were respectively added to the 5′ and 3′ terminals of the CDS sequence of the gene. A DNA fragment of the KJ541960 gene was cleaved through NdeI/EcoRI treatment from the synthesized pEX-A2J2-KJ541960 plasmid, and was linked to pET-22b(+) vector, similarly having been subjected to NdeI/EcoRI treatment, with Ligation-Convenience Kit, and thus pET-22b-KJ541960 was produced (FIG. 13). This expression construct was co-introduced into BL21 star (DE3) strain together with pACYC-del31_TaCPR to be used for protein expression.

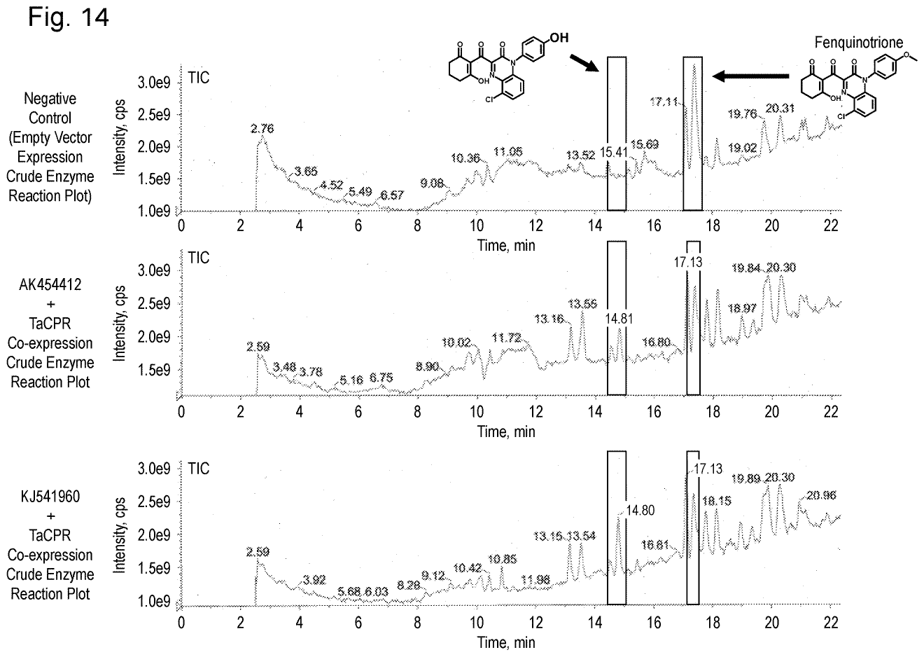

It is noted that the KJ541960 gene, synthesized as the artificial gene, was set to encode an amino acid sequence, in which a membrane anchoring region of the 35 N-terminal amino acid residues was removed, and the 6β sequence (MAKKTSS) was added as the same conditions as in the AK454412 gene. The protein expression, enzyme preparation, in vitro reaction, and analysis performed thereafter were performed by employing the test method and conditions described in Example 1. In the in vitro drug metabolism test, fenquinotrione was used as a substrate to be reacted with recombinant protein, and evaluation was performed with production of a metabolite of a demethylated product used as an index. The results are illustrated in FIG. 14. As illustrated in FIG. 14, in an empty vector crude enzyme reaction plot, used as a negative control plot, the demethylated product was not detected, but in positive control plots of AK454412 reaction plot and KJ541960 reaction plot, a parent peak of fenquinotrione was reduced, and a peak of the demethylated product appeared. Besides, the intensity of the peak of the detected demethylated product was substantially the same in both the reaction plots. It was presumed, based on this, that KJ541960 has drug metabolic capacity similar to that of AK454412.

The sequences of the primers used were as follows:

KJ541960 5′-UTR(Fd):
(SEQ ID No. 37)
5′-CAACCTGAGACCTCAAGTGTCAC-3′
KJ541960 3′-UTR (Rv):
(SEQ ID No. 38)
5′-CAGTGCTACCGCAAGATAGCTAC-3′

### [Example 6] Functional Analysis of Barley-Derived AK375492 and AK369081, Homologous Genes to AK454412

The barley-derived genes Accession Nos. AK375492 and AK369081, which are homologous genes to CYP81A6, described in Example 1 respectively have homology of 93% and 80% identity in the amino acid sequence excluding the transmembrane region to the AK454412 gene. Since these barley-derived genes thus also have high homology to the AK454412 gene, it was also verified whether or not these genes have fenquinotrione metabolic activity.

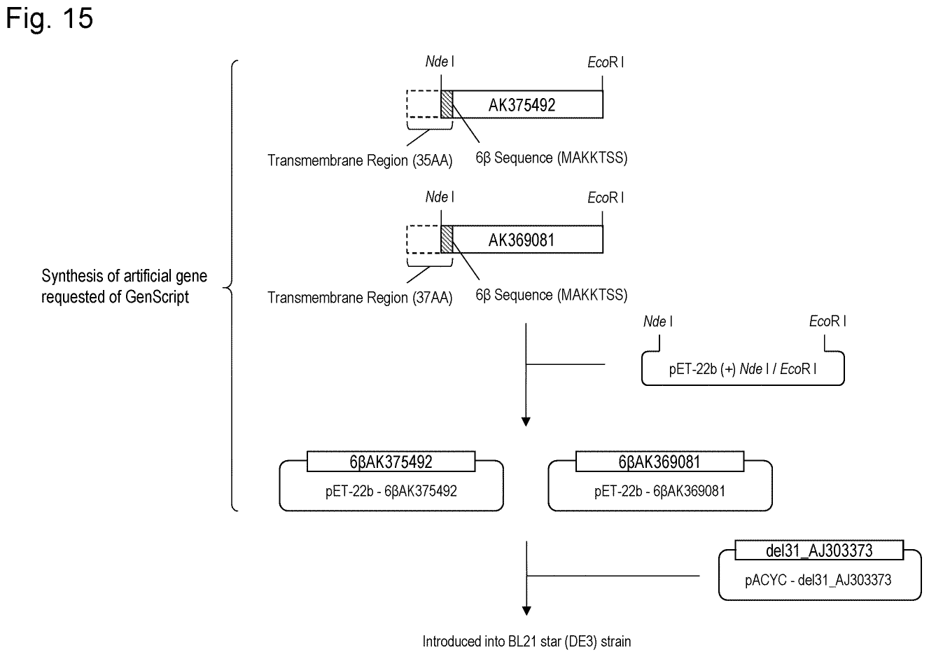

Specifically, in the same manner as in KJ541960, recombinant proteins of these genes were prepared to evaluate, by in vitro test, whether or not these have fenquinotrione metabolic activity. First, an expression vector was designed by inserting, between NdeI and EcoRI sites of pET-22b(+), a nucleotide sequence, which encoded a sequence obtained by removing the transmembrane region (AK375492: 35 N-terminal amino acid residues, AK369081: 37 N-terminal amino acid residues) of each of the genes of Accession Nos. AK375492 and AK369081, and adding the 6β sequence (MAKKTSS) to the N-terminal, and synthesis of artificial genes was requested of GenScript. Each of the synthesized expression vectors (6β AK375492 in pET-22b, 6β AK369081 in pET-22b) was co-introduced, together with pACYC-del31_TaCPR, into E. coli BL21 star (DE3) strain for protein expression (FIG. 15). The protein expression, enzyme preparation, in vitro reaction, and analysis performed thereafter were performed by employing the test method and conditions described in Example 1.

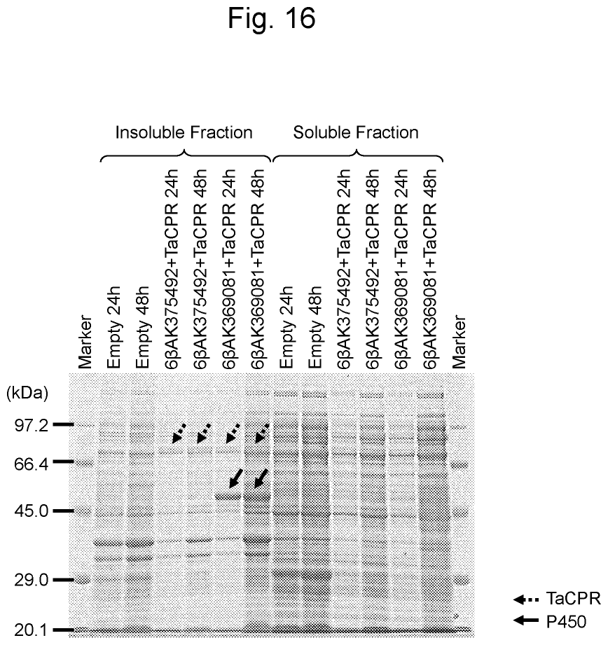

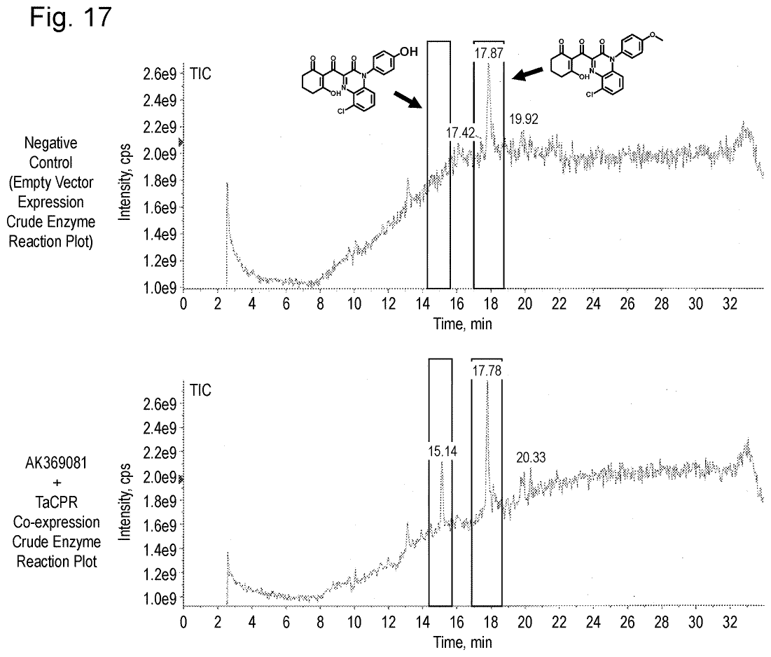

As a result of the protein expression, the expression was confirmed in an insoluble fraction in only AK369081, and the target protein was not expressed in AK375492 in this expression system (FIG. 16). Therefore, the in vitro drug metabolism test was performed on AK369081 alone. In the in vitro drug metabolism test, fenquinotrione was used as a substrate to be reacted with a recombinant protein, and evaluation was performed with generation of a metabolite of a demethylated product used as an index. The results are illustrated in FIG. 17. As illustrated in FIG. 17, a peak was not detected except for that of the parent compound in an empty vector crude enzyme reaction plot used as a negative control plot, but a peak of the demethylated product (RT=in the vicinity of 15.1) appeared in the AK369081 reaction plot. Based on this, it was presumed that AK369081 has fenquinotrione metabolic capacity similarly to AK454412.

It is noted that all publications, patents, and patent applications cited herein are incorporated herein by reference in their entirety.

1. A method for controlling weed noxious to a transgenic plant, comprising treating a field used for cultivating the transgenic plant with at least one inhibitor selected from the group consisting of an acetolactate synthase inhibitor classified as code 2 of Herbicide Resistance Action Committee (HRAC) classifications, a microtubule assembly inhibitor classified as code 3 of HRAC classifications, a photosystem II inhibitor classified as code 6 of HRAC classification, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications,
wherein the transgenic plant comprises an expression vector, and
wherein the expression vector comprises a cytochrome P450 gene encoding a protein described in any of the following (a) to (c):
(a) a protein comprising an amino acid sequence of any of SEQ ID NOS: 2, 4, 6, and 8;
(b) a protein comprising an amino acid sequence having 80% or more identity to the amino acid sequence of any of SEQ ID NOS: 2, 4, 6, and 8, and having metabolic activity against a microtubule assembly inhibitor classified as code 3 of HRAC classifications, a carotenoid biosynthesis inhibitor in phytoene desaturase (PDS) classified as code 12 of HRAC classifications, a protoporphyrinogen oxidase inhibitor classified as code 14 of HRAC classifications, and a dihydropteroate synthase inhibitor classified as code 18 of HRAC classifications; and
(c) a protein comprising an amino acid sequence encoded by a polynucleotide that hybridizes, under stringent conditions, with a polynucleotide consisting of a nucleotide sequence complementary to a nucleotide sequence of any of SEQ ID NO: 1, 3, 5, and 7, and having metabolic activity against a microtubule assembly inhibitor classified as code 3 of HRAC classifications, a carotenoid biosynthesis inhibitor in phytoene desaturase (PDS) classified as code 12 of HRAC classifications, a protoporphyrinogen oxidase inhibitor classified as code 14 of HRAC classifications, and a dihydropteroate synthase inhibitor classified as code 18 of HRAC classifications.

2. The method according to claim 1, wherein the protein consists of an amino acid sequence of (d) or (e):
(d) an amino acid sequence of any of SEQ ID NOS: 10, 12, 14, and 16; or
(e) an amino acid sequence having 80% or more identity to the amino acid sequence of any of SEQ ID NOS: 10, 12, 14, and 16.

3. The method according to claim 1, wherein the protein has metabolic activity against an acetolactate synthase inhibitor classified as code 2 of HRAC classifications, a microtubule assembly inhibitor classified as code 3 of HRAC classifications, a photosystem II inhibitor classified as code 6 of HRAC classification, a carotenoid biosynthesis inhibitor in phytoene desaturase (PDS) classified as code 12 of HRAC classifications, a protoporphyrinogen oxidase inhibitor classified as code 14 of HRAC classifications, a dihydropteroate synthase inhibitor classified as code 18 of HRAC classifications, and a 4-hydroxyphenylpyruvate dioxygenase inhibitor classified as code 27 of HRAC classifications.

4. The method according to claim 1, wherein the transgenic plant is a plant body, a plant organ, a plant tissue, or a cultured plant cell.


SEQUENCE LISTING (39 sequences)

SEQ ID NO: 1 - DNA, 1452 bp, Triticum aestivum
  source 1..1452: mol_type = genomic DNA
  CDS 1..1452
ctgccgccgagccccccggccatcccgttcctcggccacctccacctcctggagaagccgttccatgccgcgctgcgccgcctcgccgcgcgcctcggcccggtcttctcgctgcggctcggctcgcgccgcgccgttgtggtctcctcggcggagtgcgccagggagtgcttcacggagcacgacgtgacgttcgccaaccggccccggttcccctcgcagctgctcgtctccttcgacggcgccgcgctcgtcacgtccagctacggcccgcactggcgcaacctccgccgcgtcgccgccgtgcagctgctctccgcgcaccgcgtcgcctgcatgtctggcgtcatcgccggcgaggtgcgcgccatggcgcgccggctgttccgcgccgccgcggcgtcccccggcggcgatggcgccgcgcgggtccagctgaagcggaggctcttcgagctctccctcagcgtgctcatggagaccatcgcccagaccaaggcgacccggtcggaggccgacgccgacacggacatgtccgtggaggcccaggagttcaagaaggtggtggacgagctcatcccgtacctcggcgccgccaacacgtgggattacctgccggtgttgcggtggttcgacgtgttcggcgtgaggaacaagatcctggccgccgtgagcaggagggacgccttcatgctgcgtctcatcgacaacgagcgccggaggctcgacgacgctggcaccgaaggcgacaagaagagcatgatcgccgtgctcctcaatctgcagaagacggagccggaggtgtacgccgataccatgatcacggctctctgcgcgaatttatttggggccggaacggagaccacgtcgacgacgacggagtgggcgatgtcgctgctgctgaaccacccggcggcgctcaaaaaggcgcaggccgagatcgacgcggccgtgggcacctcccggctggtgaccgccgacgacgtgccacggctggcctacctgcagtgcatcgtgagcgagacgctgcggctgtatccggcggcgccgatgctgctgccgcacgagtcctcggcagactgcaaggtgggcggctacaacgtgccgagcggcacgatgctgatggtgaacgcgtacgccatccaccgggacccggcggcgtgggagcggccgctggagttcgtcccggagcggttcgaggacgggaaggccgaggggcggttcatgatcccgttcgggatgggccgccggcggtgccccggggagacgctggcgctgcggaccatcggcatggtgctggccacgctggtgcagtgcttcgactgggagcgcgtcgacggcgcggaggtggacatgacggagggcggcgggctcaccatccccaaggtcgtgccactcgaggccgtgtgcaggccgcgcccggccatgcgcgacgtgcttcagagcctctga

SEQ ID NO: 2 - protein, 483 aa, Triticum aestivum
LPPSPPAIPFLGHLHLLEKPFHAALRRLAARLGPVFSLRLGSRRAVVVSSAECARECFTEHDVTFANRPRFPSQLLVSFDGAALVTSSYGPHWRNLRRVAAVQLLSAHRVACMSGVIAGEVRAMARRLFRAAAASPGGDGAARVQLKRRLFELSLSVLMETIAQTKATRSEADADTDMSVEAQEFKKVVDELIPYLGAANTWDYLPVLRWFDVFGVRNKILAAVSRRDAFMLRLIDNERRRLDDAGTEGDKKSMIAVLLNLQKTEPEVYADTMITALCANLFGAGTETTSTTTEWAMSLLLNHPAALKKAQAEIDAAVGTSRLVTADDVPRLAYLQCIVSETLRLYPAAPMLLPHESSADCKVGGYNVPSGTMLMVNAYAIHRDPAAWERPLEFVPERFEDGKAEGRFMIPFGMGRRRCPGETLALRTIGMVLATLVQCFDWERVDGAEVDMTEGGGLTIPKVVPLEAVCRPRPAMRDVLQSL

SEQ ID NO: 3 - DNA, 1452 bp, Triticum aestivum
  source 1..1452: mol_type = genomic DNA
  CDS 1..1452
ctgccgccgagcccccaggccatcccgttcctcggccacctccacctcctggagaagccgttccacgccgctctgtgccgcctcgccgagcgcctcggcccggtcttctcgctgcgcctcggctcgcgccgcgccgtggtcgtctcctcggcggagtgcgccagggagtgcttcacggagcacgacgtgatcttcgccgaccggccccagttcccctcgcagctgctcgtgtccttcgacggcatcgcgctctccacgtccagctacggcccgcactggcgcaacctccgccgcgtcgccgccgtgcagctgctctctgcgcaccgcgtcgcctgcatgtccggcgtgatcgggggcgaggtgcgcgccatggcgcgccggctcttccgcgccgccgcggcgtcccccggtggggacggcgccgcgcgggtccagctgaagcggaggctcttcgagctctccctcagcgtgctcatggaaaccatcgcccagaccaaggcgacccggtcggaggccgacgccgacacggacatgtcagaggaggcccaggagttcaagaaggtggtggacgagctcatcccgtacctcggcgccgccaacacgtgggattacctgccggtgttgcggtggttcgacgtgttcggcgtgaggaacaagatcctggccgccgtgagcaggagggacgccttcctgcatcgtctcatcgacaacgagcgccggaggctcgacaacgccggcaccgaaggcgacaagaagagcatgatcgccgtgctcctcaatctgcagaagacggagccggaggtgtacaccgataccatgatcacggctctctgcgcgaatttattcggggccggaacggagaccacgtcgacgacgacggagtgggcgatgtcgctgctgctgaaccacccggcggcgctgaggaaggcgcaggccgagatcgacgcggccgtggggacctcccggctggtgaccgccgacgacgtgccccggctggcctacctgcagtgcatcgtgagcgagacgctgcggctgtacccggcggcgccgatgctgctgccgcaccagtcctcggcggactgcaaggtcggcggctacaacgtgccgagcggcacgatgctgatggtgaacgcgtacgccatccaccgggacccggcggcgtgggagcggccgctggagttcgtcccggagcggttcgagaacgggaaggccgaggggcggttcatgatcccgttcgggatgggccggcggcggtgccccggggagacgctggcgctgcggaccattggcatggtgctggccacgctggtgcagtgcttcgactgggagcgcgtggacggcgcggaggtggacatgacggagggcggagggctcaccatccccaaggtcgtgccgctcgaggccgtgtgcaggccgcgcccggccatgcgcgacgtgcttcagagcctctga

SEQ ID NO: 4 - protein, 483 aa, Triticum aestivum
LPPSPQAIPFLGHLHLLEKPFHAALCRLAERLGPVFSLRLGSRRAVVVSSAECARECFTEHDVIFADRPQFPSQLLVSFDGIALSTSSYGPHWRNLRRVAAVQLLSAHRVACMSGVIGGEVRAMARRLFRAAAASPGGDGAARVQLKRRLFELSLSVLMETIAQTKATRSEADADTDMSEEAQEFKKVVDELIPYLGAANTWDYLPVLRWFDVFGVRNKILAAVSRRDAFLHRLIDNERRRLDNAGTEGDKKSMIAVLLNLQKTEPEVYTDTMITALCANLFGAGTETTSTTTEWAMSLLLNHPAALRKAQAEIDAAVGTSRLVTADDVPRLAYLQCIVSETLRLYPAAPMLLPHQSSADCKVGGYNVPSGTMLMVNAYAIHRDPAAWERPLEFVPERFENGKAEGRFMIPFGMGRRRCPGETLALRTIGMVLATLVQCFDWERVDGAEVDMTEGGGLTIPKVVPLEAVCRPRPAMRDVLQSL

SEQ ID NO: 5 - DNA, 1446 bp, Hordeum vulgare
  source 1..1446: mol_type = genomic DNA
  CDS 1..1446
ctgccgccgagccccccggccgtcccgttcttcggccacctccacctcgtggagaagccgttgcacgccgcactgtgccgcctcggggcgcgccatgggccggtcttctcgcttcggctcggcgcgcgcaacgccgtggtggtgtcctcgccggcgtgcgccagggagtgcttcacggaccacgacgtggccttcgccaaccggccccagttcccctcgcagatgctcgtgtcctacggcggcacctcgctcgtcagctccagctacggcccgcactggcgcaacctccgccgcgtcgccgccgtgcgcctgctctccgcgcatcgcgtcgccggcatgtcgggcgtcatcgccgccgaggtgcgcgccatggcgcgccgtctgtaccgcgccgccgcggcgtcccccggcggcgccgcgcgcgtggagctcaagcggagcctgttcgagctctccctgagcgtgctcatggagactatcgcgcggaccaagggcacccgctcggaggcggacgccgacacggacatgtcgctggaggcgcaggagttcaagcaggtggtggacgagatcatcccgctcatcggcgcggccaacctgtgggactacctgccggtgatgcggtggttcgacgtgtccggcgtgaggagcaggatcctggccacggtgagcaggagggacgccttcctccaccggctcattgacgccgagcggcggaggatggaagagggcggcgacgagggcgagaagaagagcatgattgccgtgctcctcactctgcaaaagacggagccggagctgtacactgatcagatgatcatcgctctgtgtgcgaatatgtttgtggccggaacagagaccacctcaaccacgatagaatgggcgatgtcgctgctgctgaaccacccggcggcgctcaagaaggcccaggccgagatcgacgcgtccatcgggacctcccgcatggttgccgccgacgacgtgccccgcctcagctacctccagtgcatcatcaacgagacgctacgcatgtacccggcggcgccgctgctgctgccgcacgagtcctccgccgactgcaaggtcggcggctacgacgtgccgagcggcaccatgctgatcgtgaacgcgtacgccatccacagggacccggcgacgtgggaggacccgacggcgttccggccggagcggttcgaggacggcaagggcgacgggctgctgctgatgccgttcgggatggggcggcggaggtgccccggcgaggcgctggcgctgcagacggtcggggttgttctcgggatgctggtgcagtgcttcgactgggatcgggtggacggcgtggaggtggacatgacggagggggtggggatcaccatgcccaagtccgtggctttggaggccgtgtgtaggccgcgtgctgccatgcgcgatgtccttcacaagctgtga

SEQ ID NO: 6 - protein, 481 aa, Hordeum vulgare
LPPSPPAVPFFGHLHLVEKPLHAALCRLGARHGPVFSLRLGARNAVVVSSPACARECFTDHDVAFANRPQFPSQMLVSYGGTSLVSSSYGPHWRNLRRVAAVRLLSAHRVAGMSGVIAAEVRAMARRLYRAAAASPGGAARVELKRSLFELSLSVLMETIARTKGTRSEADADTDMSLEAQEFKQVVDEIIPLIGAANLWDYLPVMRWFDVSGVRSRILATVSRRDAFLHRLIDAERRRMEEGGDEGEKKSMIAVLLTLQKTEPELYTDQMIIALCANMFVAGTETTSTTIEWAMSLLLNHPAALKKAQAEIDASIGTSRMVAADDVPRLSYLQCIINETLRMYPAAPLLLPHESSADCKVGGYDVPSGTMLIVNAYAIHRDPATWEDPTAFRPERFEDGKGDGLLLMPFGMGRRRCPGEALALQTVGVVLGMLVQCFDWDRVDGVEVDMTEGVGITMPKSVALEAVCRPRAAMRDVLHKL

SEQ ID NO: 7 - DNA, 1452 bp, Hordeum vulgare
  source 1..1452: mol_type = genomic DNA
  CDS 1..1452
ctgccgccgagccccaggcccatcccgttcctcggccacctccacctcctggagaagccgttccacgtcgctctgtgccgcctcgccgcgcgcctcggcccggtcttctcgctgcggctcggctcgcgccgcgccgtggtggtgtcctcggcggactgcgccagggagtgcttcacggagcacgacgtgatcttcgccaacaggccccagttcccctcgcagctgctcgtgtccttcgacggcaccgcgctctccacgtccagctacggcccgcactggcgcaacctccgtcgcgtcgccgccgtgcagctgctctccgcgcaccgcgtcgcctgcatgtccggcgtcatcgccggcgaggtgcgcgccatggcccgccggctcttccgctccgccgaggcgtcccccggcggcggtggcgccgcgcgggtccagctgaagcggaggctgttcgagctctcattgagcgtgctcatggagaccatcgctcagaccaaggggacccggtcggaggccgacgccgacacggacatgtccgtggaggcccaggagttcaagaaggtggtggacgagatcatcccgtacctcggcgccgccaacacgtgggactacctgccggtggtgcggtggttcgacgtgttcggcgtcaggaacaagatcctggccgccgtgagcaggagggacgccttcctccatcgtctcatcgacgccgagcggaggaggctggacggcggcggcgccgaagccgacaagaagagcatgatcgccgtgctgctcactctgcagaagacggagccggaggtgtacaccgatactatgatcacggctctctgctcgaatttatttggggccggaacggagaccacgtcgacgacgacggagtgggccatgtcgctgcttctgaaccacccggcggcgctgaggaaggcgcaggccgagatcgacgcggccgtgggcacctcccggctggtgaccgccgacgacgtgccccggctcgcctacctgcagtgcatcgtgagcgagacgctgcggctgtacccggcgacgccgatgctgctgccgcaccaatcgtcggcggactgcaaggtgggcggctacaacgtgccgagcggcacgatgctgatggtgaacgcgtacgccatccaccgcgacccggcggcgtgggagcggccgctggagttcgtcccggagcggttcgaggacggcaaggccgaggggcggttcatgatcccgttcgggatgggccgccggcggtgccccggggagacgctggcgctgcggaccatcggcatggtgctggccacgctggtgcagtgcttcgactgggaccgcgtcgacggcaaggaggtggacatgacggagagcggcgggctcaccatccccaaggccgtgccgctcgaggccgtctgcaggccgcgcgcggccatgcgcgacgtgctccagagcctctga

SEQ ID NO: 8 - protein, 483 aa, Hordeum vulgare
LPPSPRPIPFLGHLHLLEKPFHVALCRLAARLGPVFSLRLGSRRAVVVSSADCARECFTEHDVIFANRPQFPSQLLVSFDGTALSTSSYGPHWRNLRRVAAVQLLSAHRVACMSGVIAGEVRAMARRLFRSAEASPGGGGAARVQLKRRLFELSLSVLMETIAQTKGTRSEADADTDMSVEAQEFKKVVDEIIPYLGAANTWDYLPVVRWFDVFGVRNKILAAVSRRDAFLHRLIDAERRRLDGGGAEADKKSMIAVLLTLQKTEPEVYTDTMITALCSNLFGAGTETTSTTTEWAMSLLLNHPAALRKAQAEIDAAVGTSRLVTADDVPRLAYLQCIVSETLRLYPATPMLLPHQSSADCKVGGYNVPSGTMLMVNAYAIHRDPAAWERPLEFVPERFEDGKAEGRFMIPFGMGRRRCPGETLALRTIGMVLATLVQCFDWDRVDGKEVDMTESGGLTIPKAVPLEAVCRPRAAMRDVLQSL

SEQ ID NO: 9 - DNA, 1551 bp, Triticum aestivum
  source 1..1551: mol_type = genomic DNA
  CDS 1..1551
atggataaggcgtacattgccatcctcaccatcgccttcctcttcctgatccactacgttctgggcaatggccggcgcggcggcaagggcgccgcgcagctgccgccgagccccccggccatcccgttcctcggccacctccacctcctggagaagccgttccatgccgcgctgcgccgcctcgccgcgcgcctcggcccggtcttctcgctgcggctcggctcgcgccgcgccgttgtggtctcctcggcggagtgcgccagggagtgcttcacggagcacgacgtgacgttcgccaaccggccccggttcccctcgcagctgctcgtctccttcgacggcgccgcgctcgtcacgtccagctacggcccgcactggcgcaacctccgccgcgtcgccgccgtgcagctgctctccgcgcaccgcgtcgcctgcatgtctggcgtcatcgccggcgaggtgcgcgccatggcgcgccggctgttccgcgccgccgcggcgtcccccggcggcgatggcgccgcgcgggtccagctgaagcggaggctcttcgagctctccctcagcgtgctcatggagaccatcgcccagaccaaggcgacccggtcggaggccgacgccgacacggacatgtccgtggaggcccaggagttcaagaaggtggtggacgagctcatcccgtacctcggcgccgccaacacgtgggattacctgccggtgttgcggtggttcgacgtgttcggcgtgaggaacaagatcctggccgccgtgagcaggagggacgccttcatgctgcgtctcatcgacaacgagcgccggaggctcgacgacgctggcaccgaaggcgacaagaagagcatgatcgccgtgctcctcaatctgcagaagacggagccggaggtgtacgccgataccatgatcacggctctctgcgcgaatttatttggggccggaacggagaccacgtcgacgacgacggagtgggcgatgtcgctgctgctgaaccacccggcggcgctcaaaaaggcgcaggccgagatcgacgcggccgtgggcacctcccggctggtgaccgccgacgacgtgccacggctggcctacctgcagtgcatcgtgagcgagacgctgcggctgtatccggcggcgccgatgctgctgccgcacgagtcctcggcagactgcaaggtgggcggctacaacgtgccgagcggcacgatgctgatggtgaacgcgtacgccatccaccgggacccggcggcgtgggagcggccgctggagttcgtcccggagcggttcgaggacgggaaggccgaggggcggttcatgatcccgttcgggatgggccgccggcggtgccccggggagacgctggcgctgcggaccatcggcatggtgctggccacgctggtgcagtgcttcgactgggagcgcgtcgacggcgcggaggtggacatgacggagggcggcgggctcaccatccccaaggtcgtgccactcgaggccgtgtgcaggccgcgcccggccatgcgcgacgtgcttcagagcctctga

SEQ ID NO: 10 - protein, 516 aa, Triticum aestivum
MDKAYIAILTIAFLFLIHYVLGNGRRGGKGAAQLPPSPPAIPFLGHLHLLEKPFHAALRRLAARLGPVFSLRLGSRRAVVVSSAECARECFTEHDVTFANRPRFPSQLLVSFDGAALVTSSYGPHWRNLRRVAAVQLLSAHRVACMSGVIAGEVRAMARRLFRAAAASPGGDGAARVQLKRRLFELSLSVLMETIAQTKATRSEADADTDMSVEAQEFKKVVDELIPYLGAANTWDYLPVLRWFDVFGVRNKILAAVSRRDAFMLRLIDNERRRLDDAGTEGDKKSMIAVLLNLQKTEPEVYADTMITALCANLFGAGTETTSTTTEWAMSLLLNHPAALKKAQAEIDAAVGTSRLVTADDVPRLAYLQCIVSETLRLYPAAPMLLPHESSADCKVGGYNVPSGTMLMVNAYAIHRDPAAWERPLEFVPERFEDGKAEGRFMIPFGMGRRRCPGETLALRTIGMVLATLVQCFDWERVDGAEVDMTEGGGLTIPKVVPLEAVCRPRPAMRDVLQSL

SEQ ID NO: 11 - DNA, 1557 bp, Triticum aestivum
  source 1..1557: mol_type = genomic DNA
  CDS 1..1557
atggataaggcatatatcgccatcctctccttcgccttcctcttcctgctccactacattctaggcaaggtcagcaatggcaggcgcagcaagggcgccgtccagctgccgccgagcccccaggccatcccgttcctcggccacctccacctcctggagaagccgttccacgccgctctgtgccgcctcgccgagcgcctcggcccggtcttctcgctgcgcctcggctcgcgccgcgccgtggtcgtctcctcggcggagtgcgccagggagtgcttcacggagcacgacgtgatcttcgccgaccggccccagttcccctcgcagctgctcgtgtccttcgacggcatcgcgctctccacgtccagctacggcccgcactggcgcaacctccgccgcgtcgccgccgtgcagctgctctctgcgcaccgcgtcgcctgcatgtccggcgtgatcgggggcgaggtgcgcgccatggcgcgccggctcttccgcgccgccgcggcgtcccccggtggggacggcgccgcgcgggtccagctgaagcggaggctcttcgagctctccctcagcgtgctcatggaaaccatcgcccagaccaaggcgacccggtcggaggccgacgccgacacggacatgtcagaggaggcccaggagttcaagaaggtggtggacgagctcatcccgtacctcggcgccgccaacacgtgggattacctgccggtgttgcggtggttcgacgtgttcggcgtgaggaacaagatcctggccgccgtgagcaggagggacgccttcctgcatcgtctcatcgacaacgagcgccggaggctcgacaacgccggcaccgaaggcgacaagaagagcatgatcgccgtgctcctcaatctgcagaagacggagccggaggtgtacaccgataccatgatcacggctctctgcgcgaatttattcggggccggaacggagaccacgtcgacgacgacggagtgggcgatgtcgctgctgctgaaccacccggcggcgctgaggaaggcgcaggccgagatcgacgcggccgtggggacctcccggctggtgaccgccgacgacgtgccccggctggcctacctgcagtgcatcgtgagcgagacgctgcggctgtacccggcggcgccgatgctgctgccgcaccagtcctcggcggactgcaaggtcggcggctacaacgtgccgagcggcacgatgctgatggtgaacgcgtacgccatccaccgggacccggcggcgtgggagcggccgctggagttcgtcccggagcggttcgagaacgggaaggccgaggggcggttcatgatcccgttcgggatgggccggcggcggtgccccggggagacgctggcgctgcggaccattggcatggtgctggccacgctggtgcagtgcttcgactgggagcgcgtggacggcgcggaggtggacatgacggagggcggagggctcaccatccccaaggtcgtgccgctcgaggccgtgtgcaggccgcgcccggccatgcgcgacgtgcttcagagcctctga

SEQ ID NO: 12 - protein, 518 aa, Triticum aestivum
MDKAYIAILSFAFLFLLHYILGKVSNGRRSKGAVQLPPSPQAIPFLGHLHLLEKPFHAALCRLAERLGPVFSLRLGSRRAVVVSSAECARECFTEHDVIFADRPQFPSQLLVSFDGIALSTSSYGPHWRNLRRVAAVQLLSAHRVACMSGVIGGEVRAMARRLFRAAAASPGGDGAARVQLKRRLFELSLSVLMETIAQTKATRSEADADTDMSEEAQEFKKVVDELIPYLGAANTWDYLPVLRWFDVFGVRNKILAAVSRRDAFLHRLIDNERRRLDNAGTEGDKKSMIAVLLNLQKTEPEVYTDTMITALCANLFGAGTETTSTTTEWAMSLLLNHPAALRKAQAEIDAAVGTSRLVTADDVPRLAYLQCIVSETLRLYPAAPMLLPHQSSADCKVGGYNVPSGTMLMVNAYAIHRDPAAWERPLEFVPERFENGKAEGRFMIPFGMGRRRCPGETLALRTIGMVLATLVQCFDWERVDGAEVDMTEGGGLTIPKVVPLEAVCRPRPAMRDVLQSL

SEQ ID NO: 13 - DNA, 1557 bp, Hordeum vulgare
  source 1..1557: mol_type = genomic DNA
  CDS 1..1557
atggataaggcatccattgccgtcctctccttggccttcctcttcctgctccactacattctgggcaagaggagcgatggcaggcgcggcaagggcaagggcgccgtgcagctgccgccgagccccccggccgtcccgttcttcggccacctccacctcgtggagaagccgttgcacgccgcactgtgccgcctcggggcgcgccatgggccggtcttctcgcttcggctcggcgcgcgcaacgccgtggtggtgtcctcgccggcgtgcgccagggagtgcttcacggaccacgacgtggccttcgccaaccggccccagttcccctcgcagatgctcgtgtcctacggcggcacctcgctcgtcagctccagctacggcccgcactggcgcaacctccgccgcgtcgccgccgtgcgcctgctctccgcgcatcgcgtcgccggcatgtcgggcgtcatcgccgccgaggtgcgcgccatggcgcgccgtctgtaccgcgccgccgcggcgtcccccggcggcgccgcgcgcgtggagctcaagcggagcctgttcgagctctccctgagcgtgctcatggagactatcgcgcggaccaagggcacccgctcggaggcggacgccgacacggacatgtcgctggaggcgcaggagttcaagcaggtggtggacgagatcatcccgctcatcggcgcggccaacctgtgggactacctgccggtgatgcggtggttcgacgtgtccggcgtgaggagcaggatcctggccacggtgagcaggagggacgccttcctccaccggctcattgacgccgagcggcggaggatggaagagggcggcgacgagggcgagaagaagagcatgattgccgtgctcctcactctgcaaaagacggagccggagctgtacactgatcagatgatcatcgctctgtgtgcgaatatgtttgtggccggaacagagaccacctcaaccacgatagaatgggcgatgtcgctgctgctgaaccacccggcggcgctcaagaaggcccaggccgagatcgacgcgtccatcgggacctcccgcatggttgccgccgacgacgtgccccgcctcagctacctccagtgcatcatcaacgagacgctacgcatgtacccggcggcgccgctgctgctgccgcacgagtcctccgccgactgcaaggtcggcggctacgacgtgccgagcggcaccatgctgatcgtgaacgcgtacgccatccacagggacccggcgacgtgggaggacccgacggcgttccggccggagcggttcgaggacggcaagggcgacgggctgctgctgatgccgttcgggatggggcggcggaggtgccccggcgaggcgctggcgctgcagacggtcggggttgttctcgggatgctggtgcagtgcttcgactgggatcgggtggacggcgtggaggtggacatgacggagggggtggggatcaccatgcccaagtccgtggctttggaggccgtgtgtaggccgcgtgctgccatgcgcgatgtccttcacaagctgtga

SEQ ID NO: 14 - protein, 518 aa, Hordeum vulgare
MDKASIAVLSLAFLFLLHYILGKRSDGRRGKGKGAVQLPPSPPAVPFFGHLHLVEKPLHAALCRLGARHGPVFSLRLGARNAVVVSSPACARECFTDHDVAFANRPQFPSQMLVSYGGTSLVSSSYGPHWRNLRRVAAVRLLSAHRVAGMSGVIAAEVRAMARRLYRAAAASPGGAARVELKRSLFELSLSVLMETIARTKGTRSEADADTDMSLEAQEFKQVVDEIIPLIGAANLWDYLPVMRWFDVSGVRSRILATVSRRDAFLHRLIDAERRRMEEGGDEGEKKSMIAVLLTLQKTEPELYTDQMIIALCANMFVAGTETTSTTIEWAMSLLLNHPAALKKAQAEIDASIGTSRMVAADDVPRLSYLQCIINETLRMYPAAPLLLPHESSADCKVGGYDVPSGTMLIVNAYAIHRDPATWEDPTAFRPERFEDGKGDGLLLMPFGMGRRRCPGEALALQTVGVVLGMLVQCFDWDRVDGVEVDMTEGVGITMPKSVALEAVCRPRAAMRDVLHKL

SEQ ID NO: 15 - DNA, 1557 bp, Hordeum vulgare
  source 1..1557: mol_type = genomic DNA
  CDS 1..1557
atggataaggcatacattgccatcctctccttcactttcctcttcctgctccactacattctaggcaaggtcagcaatggcaggcgcagcaagggtgacgtccagctgccgccgagccccaggcccatcccgttcctcggccacctccacctcctggagaagccgttccacgtcgctctgtgccgcctcgccgcgcgcctcggcccggtcttctcgctgcggctcggctcgcgccgcgccgtggtggtgtcctcggcggactgcgccagggagtgcttcacggagcacgacgtgatcttcgccaacaggccccagttcccctcgcagctgctcgtgtccttcgacggcaccgcgctctccacgtccagctacggcccgcactggcgcaacctccgtcgcgtcgccgccgtgcagctgctctccgcgcaccgcgtcgcctgcatgtccggcgtcatcgccggcgaggtgcgcgccatggcccgccggctcttccgctccgccgaggcgtcccccggcggcggtggcgccgcgcgggtccagctgaagcggaggctgttcgagctctcattgagcgtgctcatggagaccatcgctcagaccaaggggacccggtcggaggccgacgccgacacggacatgtccgtggaggcccaggagttcaagaaggtggtggacgagatcatcccgtacctcggcgccgccaacacgtgggactacctgccggtggtgcggtggttcgacgtgttcggcgtcaggaacaagatcctggccgccgtgagcaggagggacgccttcctccatcgtctcatcgacgccgagcggaggaggctggacggcggcggcgccgaagccgacaagaagagcatgatcgccgtgctgctcactctgcagaagacggagccggaggtgtacaccgatactatgatcacggctctctgctcgaatttatttggggccggaacggagaccacgtcgacgacgacggagtgggccatgtcgctgcttctgaaccacccggcggcgctgaggaaggcgcaggccgagatcgacgcggccgtgggcacctcccggctggtgaccgccgacgacgtgccccggctcgcctacctgcagtgcatcgtgagcgagacgctgcggctgtacccggcgacgccgatgctgctgccgcaccaatcgtcggcggactgcaaggtgggcggctacaacgtgccgagcggcacgatgctgatggtgaacgcgtacgccatccaccgcgacccggcggcgtgggagcggccgctggagttcgtcccggagcggttcgaggacggcaaggccgaggggcggttcatgatcccgttcgggatgggccgccggcggtgccccggggagacgctggcgctgcggaccatcggcatggtgctggccacgctggtgcagtgcttcgactgggaccgcgtcgacggcaaggaggtggacatgacggagagcggcgggctcaccatccccaaggccgtgccgctcgaggccgtctgcaggccgcgcgcggccatgcgcgacgtgctccagagcctctga

SEQ ID NO: 16 - protein, 518 aa, Hordeum vulgare
MDKAYIAILSFTFLFLLHYILGKVSNGRRSKGDVQLPPSPRPIPFLGHLHLLEKPFHVALCRLAARLGPVFSLRLGSRRAVVVSSADCARECFTEHDVIFANRPQFPSQLLVSFDGTALSTSSYGPHWRNLRRVAAVQLLSAHRVACMSGVIAGEVRAMARRLFRSAEASPGGGGAARVQLKRRLFELSLSVLMETIAQTKGTRSEADADTDMSVEAQEFKKVVDEIIPYLGAANTWDYLPVVRWFDVFGVRNKILAAVSRRDAFLHRLIDAERRRLDGGGAEADKKSMIAVLLTLQKTEPEVYTDTMITALCSNLFGAGTETTSTTTEWAMSLLLNHPAALRKAQAEIDAAVGTSRLVTADDVPRLAYLQCIVSETLRLYPATPMLLPHQSSADCKVGGYNVPSGTMLMVNAYAIHRDPAAWERPLEFVPERFEDGKAEGRFMIPFGMGRRRCPGETLALRTIGMVLATLVQCFDWDRVDGKEVDMTESGGLTIPKAVPLEAVCRPRAAMRDVLQSL

SEQ ID NO: 17 - DNA, 23 bp, synthetic construct
  misc_feature 1..23: note = Synthetic DNA
  source 1..23: mol_type = other DNA
caacctgagacctcaagtgtcac

SEQ ID NO: 18 - DNA, 21 bp, synthetic construct
  misc_feature 1..21: note = Synthetic DNA
  source 1..21: mol_type = other DNA
gtactacctggatccacgagc

SEQ ID NO: 19 - DNA, 35 bp, synthetic construct
  misc_feature 1..35: note = Synthetic DNA
  source 1..35: mol_type = other DNA
aaggagatatacatatggataaggcgtacattgcc

SEQ ID NO: 20 - DNA, 35 bp, synthetic construct
  misc_feature 1..35: note = Synthetic DNA
  source 1..35: mol_type = other DNA
gtggtggtgctcgagtcagaggctctgaagcacgt

SEQ ID NO: 21 - DNA, 2091 bp, Triticum aestivum
  source 1..2091: mol_type = genomic DNA
atgagggactcggcgctcgacctgctggcggcgctgctcacgggccgggccccgccggccgccgccgacggggaccagaaccgccgcctgctcgcgctgctcgccacctccctcgccgtgctcgtcggctgcggcgtcgcgctcctcttccgccgctcctcctccggcgccgcgccgctcgcccacaagtccgccgcggccaagcccctcgcagcgaagaaggaccaggagcccgaccccgacgacggccgccagcgggtcgccctcttcttcggcacgcagaccggcaccgccgagggcttcgccaaggcgctcgcggaggaggccaaggccaggtacgacaaggccgtcttcaaggtgctcgatctggatgactatgccgcggaggacgaggagtacgaggagaagctcaagaaggagaacatcgccttcttcttcctcgcaacgtatggggacggtgagccaaccgacaatgccgccagattctacaaatggttctccgagggaaacgagaggggtgagtggttgagcaacctcaagttcggggtgtttgctcttgggaataggcagtacgagcatttcaacaaggtcggaaaggaggtcgatcagctcctcgccgaacaaggtggaaagcgcatagttcctgttggccttggagatgacgatcaatgcattgaggatgacttcaatgcctggaaggaactactgtggccagaattggacaaactgctccgcgttgaagataattcttcaacagcacaatctccttacacagctgctattccgcaatatagagttgtgcttaccaaaccagaggatgccacacatatcaacaagtctttcagtcttagcaatggtcatgtcgtctacgacagtcaacatccttgcagggcaaatgtggctgtgcgacgggagcttcacacaccagcttctgaccggtcctgcattcatttggaatttgatattgcaggaactagccttacatatgagactggagatcatgttggtgtgtacgcagaaaattcaattgagactgtagaggaggcagaaaagctattagattattcaccagatacttatttctcaatttatgctgaccaagaggatggcactccactttttgggggctctttgccacctcctttcccatctccctgtactgtgagggttgcacttgccagatatgccgatctgttgaattcacctaaaaagagtgttttacttgctttggctgctcatgcatctgatcccaaagaggccgagcggctcagacatctagcatctcctgctggaaagaaggagtattctcagtggataatcgctagtcaaagaagtctcttggaagttatttcggagtttccatcggcgaaacctccgcttggtgtcttctttgcagctattgctcctcgccttcagccgagatactactcaatatcttcctcaccaagaatggcacctacaagaattcatgtaacatgttcactagttcatgggcaaaccccaactggaaggatccataaaggagtttgttctacttggatgaagaattcaactcccttggaagagagccaagaatgtagctgggctccaatttttgtgaggcagtcaaacttcaaattgcctgctgatcccacagtgcctgttataatggtaggccctgggactggccttgcacctttcaggggcttcttacaggaaagattagctttaaaagagaccggggtggaactgggccgtgccatcctgtttttcgggtgcagaaaccgccagatggacttcatatatgaggatgagctgaacaactttgccgaatccggagccctgtctgagttggtcgtcgccttttctcgtgagggtcctacaaaggagtacgtccaacataaaatggcagagaaggctgcagagctgtggagcattgtttcccagggcggctatgtgtacgtctgcggcgacgccaagggcatggcgagagacgtgcaccgcgcgctgcacaccatagttcaggagcagggatctctggacagctccaaggcggagggctatgtgaaaaaccttcagatggaagggagatacctgagagacgtctggtaa

SEQ ID NO: 22 - DNA, 2112 bp, Triticum aestivum
  source 1..2112: mol_type = genomic DNA
atggactccgccgccgcggggatgagggactcggcgctcgacctgctggcggcgctgctcacgggccgggccccgccggccgccgccgacggggaccagaaccgccgcctgctcgcgctgctcgccacctccctcgccgtgctcgtcggctgcggcgtcgcgctcctcttccgccgctcctcctccggcgccgcgccgctcgcccacaagtccgccgcggccaagcccctcgcagcgaagaaggaccaggagcccgaccccgacgacggccgccagcgggtcgccctcttcttcggcacgcagaccggcaccgccgagggcttcgccaaggcgctcgcggaggaggccaaggccaggtacgacaaggccgtcttcaaggtgctcgatctggatgactatgccgcggaggacgaggagtacgaggagaagctcaagaaggagaacatcgccttcttcttcctcgcaacgtatggggacggtgagccaaccgacaatgccgccagattctacaaatggttctccgagggaaacgagaggggtgagtggttgagcaacctcaagttcggggtgtttgctcttgggaataggcagtacgagcatttcaacaaggtcggaaaggaggtcgatcagctcctcgccgaacaaggtggaaagcgcatagttcctgttggccttggagatgacgatcaatgcattgaggatgacttcaatgcctggaaggaactactgtggccagaattggacaaactgctccgcgttgaagataattcttcaacagcacaatctccttacacagctgctattccgcaatatagagttgtgcttaccaaaccagaggatgccacacatatcaacaagtctttcagtcttagcaatggtcatgtcgtctacgacagtcaacatccttgcagggcaaatgtggctgtgcgacgggagcttcacacaccagcttctgaccggtcctgcattcatttggaatttgatattgcaggaactagccttacatatgagactggagatcatgttggtgtgtacgcagaaaattcaattgagactgtagaggaggcagaaaagctattagattattcaccagatacttatttctcaatttatgctgaccaagaggatggcactccactttttgggggctctttgccacctcctttcccatctccctgtactgtgagggttgcacttgccagatatgccgatctgttgaattcacctaaaaagagtgttttacttgctttggctgctcatgcatctgatcccaaagaggccgagcggctcagacatctagcatctcctgctggaaagaaggagtattctcagtggataatcgctagtcaaagaagtctcttggaagttatttcggagtttccatcggcgaaacctccgcttggtgtcttctttgcagctattgctcctcgccttcagccgagatactactcaatatcttcctcaccaagaatggcacctacaagaattcatgtaacatgttcactagttcatgggcaaaccccaactggaaggatccataaaggagtttgttctacttggatgaagaattcaactcccttggaagagagccaagaatgtagctgggctccaatttttgtgaggcagtcaaacttcaaattgcctgctgatcccacagtgcctattataatggtaggccctgggactggccttgcacctttcaggggcttcttacaggaaagattagctttaaaagagaccggggtggaactgggccgtgccatcctgtttttcgggtgcagaaaccgccagatggacttcatatatgaggatgagctgaacaactttgccgaatccggagccctgtctgagttggtcgtcgccttttctcgtgagggtcctacaaaggagtacgtccaacataaaatggcagagaaggctgcagagctgtggagcattgtttcccagggcggctatgtgtacgtctgcggcgacgccaagggcatggcgagagacgtgcaccgcgcgctgcacaccatagttcaggagcagggatctctggacagctccaaggcggagggctatgtgaaaaaccttcagatggaagggagatacctgagagacgtctggtaa

SEQ ID NO: 23 - DNA, 21 bp, synthetic construct
  misc_feature 1..21: note = Synthetic DNA
  source 1..21: mol_type = other DNA
cacgcgtccgatcgaaccaac

SEQ ID NO: 24 - DNA, 26 bp, synthetic construct
  misc_feature 1..26: note = Synthetic DNA
  source 1..26: mol_type = other DNA
cgatgaatcgtcatcctctgttccac

SEQ ID NO: 25 - DNA, 35 bp, synthetic construct
  misc_feature 1..35: note = Synthetic DNA
  source 1..35: mol_type = other DNA
aaggagatatacatatggactccgccgccgcgggg

SEQ ID NO: 26 - DNA, 35 bp, synthetic construct
  misc_feature 1..35: note = Synthetic DNA
  source 1..35: mol_type = other DNA
ctttaccagactcgattaccagacgtctctcaggt

SEQ ID NO: 27 - DNA, 35 bp, synthetic construct
  misc_feature 1..35: note = Synthetic DNA
  source 1..35: mol_type = other DNA
aaggagatatacatatggctaagaagacttcttct

SEQ ID NO: 28 - DNA, 35 bp, synthetic construct; identical to SEQ ID NO: 20
  misc_feature 1..35: note = Synthetic DNA
  source 1..35: mol_type = other DNA

SEQ ID NO: 29 - DNA, 35 bp, synthetic construct
  misc_feature 1..35: note = Synthetic DNA
  source 1..35: mol_type = other DNA
aaggagatatacatatggaccagaaccgccgcctg

SEQ ID NO: 30 - DNA, 35 bp, synthetic construct
  misc_feature 1..35: note = Synthetic DNA
  source 1..35: mol_type = other DNA
tttaccagactcgagttaccagacgtctctcaggt

SEQ ID NO: 31 - DNA, 35 bp, synthetic construct
  misc_feature 1..35: note = Synthetic DNA
  source 1..35: mol_type = other DNA
tacaactacatctagaatggataaggcgtacattg

SEQ ID NO: 32 - DNA, 35 bp, synthetic construct
  misc_feature 1..35: note = Synthetic DNA
  source 1..35: mol_type = other DNA
ggggaaattcgagctctcagaggctctgaagcacg

SEQ ID NO: 33 - DNA, 22 bp, synthetic construct
  misc_feature 1..22: note = Synthetic DNA
  source 1..22: mol_type = other DNA
gctctccaacaacattgccaac

SEQ ID NO: 34 - DNA, 21 bp, synthetic construct
  misc_feature 1..21: note = Synthetic DNA
  source 1..21: mol_type = other DNA
gcttctgcctgtcacatacgc

SEQ ID NO: 35 - DNA, 20 bp, synthetic construct
  misc_feature 1..20: note = Synthetic DNA
  source 1..20: mol_type = other DNA
ctctgcgcgaatttatttgg

SEQ ID NO: 36 - DNA, 20 bp, synthetic construct
  misc_feature 1..20: note = Synthetic DNA
  source 1..20: mol_type = other DNA
tcacgatgcactgcaggtag

SEQ ID NO: 37 - DNA, 23 bp, synthetic construct
  misc_feature 1..23: note = Synthetic DNA
  source 1..23: mol_type = other DNA
caacctgagacctcaagtgtcac

SEQ ID NO: 38 - DNA, 23 bp, synthetic construct
  misc_feature 1..23: note = Synthetic DNA
  source 1..23: mol_type = other DNA
cagtgctaccgcaagatagctac

SEQ ID NO: 39 - protein, 513 aa, Oryza sativa
  source 1..513: note = Oryza sativa Indica Group
MDNAYIIAILSVAILFLLHYYLLGRGNGGAARLPPGPPAVPILGHLHLVKKPMHATMSRLAERYGPVFSLRLGSRRAVVVSSPGCARECFTEHDVTFANRPRFESQLLVSFNGAALATASYGAHWRNLRRIVAVQLLSAHRVGLMSGLIAGEVRAMVRRMYRAAAASPAGAARIQLKRRLFEVSLSVLMETIAHTKATRPETDPDTDMSVEAQEFKQVVDEIIPHIGAANLWDYLPALRWFDVFGVRRKILAAVSRRDAFLRRLIDAERRRLDDGDEGEKKSMIAVLLTLQKTEPEVYTDNMITALTANLFGAGTETTSTTSEWAMSLLLNHPDTLKKAQAEIDASVGNSRLITADDVTRLGYLQCIVRETLRLYPAAPMLLPHESSADCKVGGYNIPRGSMLLINAYAIHRDPAVWEEPEKFMPERFEDGGCDGNLLMPFGMGRRRCPGETLALRTVGLVLGTLIQCFDWERVDGVEVDMTEGGGLTIPKVVPLEAMCRPRDAMGGVLRELV